(19) United States
(12) Patent Application Publication
(10) Pub. No.: US 2020/0407393 A1
(43) Pub. Date: Dec. 31, 2020

(54) Medical Use

(71) Applicants: GLAXOSMITHKLINE INTELLECTUAL PROPERTY (NO. 2) LIMITED, Brentford, Middlesex (GB); VIIV HEALTHCARE COMPANY, Wilmington, DE (US)

(72) Inventors: Martha Alicia DE LA ROSA, Research Triangle Park, NC (US); John Franklin MILLER, Research Triangle Park, NC (US); David TEMELKOFF, Research Triangle Park, NC (US); Emile Johann VELTHUISEN, Research Triangle Park, NC (US)

(73) Assignees: GLAXOSMITHKLINE INTELLECTUAL PROPERTY (NO. 2) LIMITED, Brentford, Middlesex (GB); VIIV HEALTHCARE COMPANY, Wilmington, DE (US)

(21) Appl. No.: 16/975,732

(22) PCT Filed: Mar. 6, 2019
(86) PCT No.: PCT/IB2019/051799

§ 371 Date: Aug. 26, 2020

Related U.S. Application Data
(60) Provisional application No. 62/639,667, filed on Mar. 7, 2018.

(51) Int. Cl. C07H 19/16; A61P 31/18

(52) U.S. Cl. CPC C07H 19/16; A61P 31/18

(57) ABSTRACT

The present invention relates to compounds of formula (I) and salts thereof, pharmaceutical compositions containing such compounds and to their use in therapy.

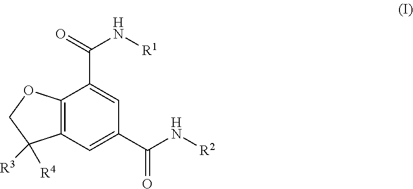

### CROSS-REFERENCE TO RELATED APPLICATIONS

The present application claims priority to U.S. Provisional Ser. No. 62/639,667, filed Mar. 7, 2018, the disclosure of which is incorporated herein by reference in its entirety.

### FIELD OF THE INVENTION

The present invention relates to compounds, pharmaceutical compositions, and methods of use thereof in connection with individuals infected with HIV.

### BACKGROUND OF THE INVENTION

Human immunodeficiency virus type 1 (HIV-1) infection leads to the contraction of acquired immune deficiency disease (AIDS). The number of cases of HIV continues to rise, and currently an estimated over thirty-five million individuals worldwide suffer from HIV infection e.g., http://www.sciencedirect.com/science/article/pii/5235230181630087X? via %3Dihub

Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Nutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

HAART therapies are often complex because a combination of different drugs must be administered often daily to the patient to avoid the rapid emergence of drug-resistant HIV-1 variants. Despite the positive impact of HAART on patient survival, drug resistance can still occur.

Current guidelines recommend that therapy includes three fully active drugs. See e.g. https://aidsinfo.nih.gov/guidelines.

Typically, first-line therapies combine two to three drugs targeting the viral enzymes reverse transcriptase and integrase. It is believed that sustained successful treatment of HIV-1-infected patients with antiretroviral drugs employ the continued development of new and improved drugs that are effective against HIV strains that have formed resistance to approved drugs. For example an individual on a regimen containing 3TC/FTC may select for the M184V mutation that reduces susceptibility to these drugs by >100 fold. See e g., https://hivdb.stanford.edu/dr-summary/resistance-notes/NRTI Another way to potentially address preventing formation of mutations is to increase patient adherence to a drug regimen. One manner that may accomplish this is by reducing the dosing frequency. For parenteral administration, it is believed to be advantageous to have drug substances with high lipophilicity in order to reduce solubility and limit the release rate within interstitial fluid. However, most nucleoside reverse transcriptase inhibitors are hydrophilic thereby potentially limiting their use as long acting parenteral agents.

There remains a need for compounds which may the shortcomings set forth above.

### SUMMARY OF THE INVENTION

In one aspect, the invention provides a compound of the formula (I):

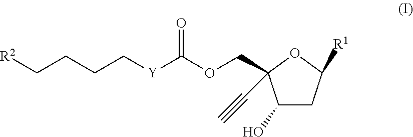

wherein:

R₁ is selected from the group consisting of:

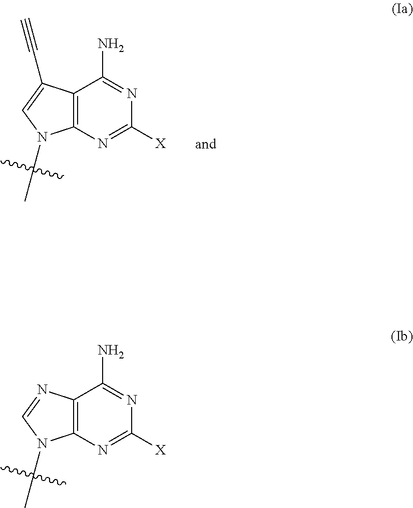

wherein:

X is selected from the group consisting of NH₂, F and Cl;

Y is selected from the group consisting of a bond, (C₁-C₁₀) alkyl, and CR³R^{3′}, wherein R³ and R^{3′} are independently selected from the group consisting of H, (C₁-C₆) alkyl, (C₁-C₆) haloalkyl, (C₂-C₁₀) alkenyl, (C₁-C₁₀) alkynyl and (C₃-C₁₄) cycloalkyl; and each of R³ and R^{3′} may be independently optionally substituted by (C₁-C₆) alkyl, Cl, F, oxo, or (C₁-C₆) alkoxy

R² is selected from the group consisting of H, (C₁-C₁₀) alkyl, (C₂-C₁₀) alkenyl, (C₂-C₁₀) alkynyl, (C₁-C₁₀) alkoxy and (C₁-C₁₀) haloalkyl; wherein each of R² may be optionally substituted by (C₁-C₆) alkyl, Cl, F, oxo, or (C₁-C₆) alkoxy or a pharmaceutically acceptable salt thereof.

In another aspect, the invention provides pharmaceutical compositions comprising a compound of Formula (I) or a pharmaceutically acceptable salt thereof and an excipient

In another aspect, the invention provides a method of treating or preventing an HIV infection in a subject at risk for developing an HIV infection, comprising administering to the subject a compound of Formula (I), or a pharmaceutically acceptable salt thereof.

In another aspect, there is provided a compound of Formula (I) or a pharmaceutically acceptable salt thereof for use in therapy.

In another aspect, there is provided a compound of Formula (I) or a pharmaceutically acceptable salt thereof for use in treating or preventing an HIV infection.

In another aspect, there is provided the use of a compound of Formula (I) or a pharmaceutically acceptable salt thereof in the manufacture of a medicament for treating or preventing an HIV infection.

These and other aspects are encompassed by the invention as set forth herein.

### BRIEF DESCRIPTION OF THE DRAWINGS

FIG. 1A illustrates an IC₅₀ cure shift from t=0 to t=48 h for EFdA.

FIG. 1B illustrates an IC₅₀ cure shift from t=0 to t=48 h for Example 5 of the present invention.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

As used herein unless otherwise specified, “alkyl” refers to a monovalent saturated aliphatic hydrocarbyl group having from 1 to 14 carbon atoms and, in some embodiments, from 1 to 6 carbon atoms. “(Cₓ-C_{y})alkyl” refers to alkyl groups having from x to y carbon atoms. The term “alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). For the purposes of the invention, alkyl may be interpreted to encompass alkylene groups defined herein.

Subject to embodiments set forth herein, “Alkylene” or “alkylene” refers to divalent e.g., saturated aliphatic hydrocarbyl groups having from 1 to 6 carbon atoms. The alkylene groups include branched and straight chain hydrocarbyl groups. For example, “(C₁-C₆)alkylene” is meant to include methylene, ethylene, propylene, 2-methypropylene, dimethylethylene, pentylene, and so forth. As such, the term “propylene” could be exemplified by the following structure:

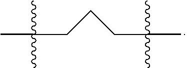 [SMILES: *CCC*]

Likewise, the term “dimethylbutylene” could be exemplified by any of the following three structures or more:

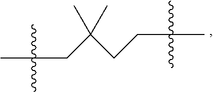 [SMILES: *CCC(C)(C)C*]

p or

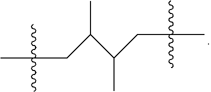 [SMILES: *CC(C)C(C)C*]

Furthermore, the term “(C₁-C₆)alkylene” is meant to include such branched chain hydrocarbyl groups as

cyclopropylmethylene, which could be exemplified by the following structure:

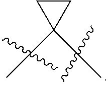 [SMILES: *C1(*)CC1]

“Alkenyl” refers to a linear or branched hydrocarbyl group having, e.g., from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, isopropylene, 1,3-butadienyl, and the like.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Alkoxy” refers to the group —O-alkyl wherein alkyl is defined herein, e.g., C₁ to C₆ alkoxy. Alkoxy includes, by way of example, methoxy, ethoxy, n-propoxy, isopropoxy, n-butoxy, t-butoxy, sec-butoxy, and n-pentoxy.

““Aryl” refers to an aromatic group of from 5 to 6 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“AUC” refers to the area under the plot of plasma concentration of drug (not logarithm of the concentration) against time after drug administration.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “cycloalkyl” includes cycloalkenyl groups, such as cyclohexenyl. Examples of cycloalkyl groups include, for instance, adamantyl, cyclopropyl, cyclobutyl, cyclohexyl, cyclopentyl, cyclooctyl, cyclopentenyl, and cyclohexenyl. Examples of cycloalkyl groups that include multiple bicycloalkyl ring systems are bicyclohexyl, bicyclopentyl, bicyclooctyl, and the like. Two such bicycloalkyl multiple ring structures are exemplified and named below:

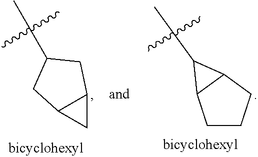 [SMILES: *C1CC2CC2C1 ; *C1C2CCCC12]

“(Cᵤ-Cᵥ)cycloalkyl” refers to cycloalkyl groups having u to v carbon atoms.

“EC₅₀” refers to the concentration of a drug that gives half-maximal response.

“IC₅₀” refers to the half-maximal inhibitory concentration of a drug. Sometimes, it is also converted to the plC₅₀ scale (−log IC₅₀), in which higher values indicate exponentially greater potency.

“Haloalkyl” refers to substitution of an alkyl group with 1 to 3 halo groups (e.g., bifluoromethyl or trifluoromethyl).

“Heteroaryl” refers to an aromatic group of from 1 to 14 carbon atoms, e.g., 5 to 6 heteroatoms selected from oxygen, nitrogen, and sulfur and includes single ring (e.g. imidazolyl) and (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, imidazolinyl, isoxazolyl, pyrrolyl, pyrazolyl, pyridazinyl, pyrimidinyl, purinyl, phthalazyl, naphthylpryidyl, benzofuranyl, tetrahydrobenzofuranyl, isobenzofuranyl, benzothiazolyl, benzoisothiazolyl, benzotriazolyl, indolyl, isoindolyl, indolizinyl, dihydroindolyl, indazolyl, indolinyl, benzoxazolyl, quinolyl, isoquinolyl, quinolizyl, quianazolyl, quinoxalyl, tetrahydroquinolinyl, isoquinolyl, quinazolinonyl, benzimidazolyl, benzisoxazolyl, benzothienyl, benzopyridazinyl, pteridinyl, carbazolyl, carbolinyl, phenanthridinyl, acridinyl, phenanthrolinyl, phenazinyl, phenoxazinyl, phenothiazinyl, and phthalimidyl.

Examples of heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholine), piperidine, pyrrolidine, and tetrahydrofuranyl.

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

“Oxo” refers to a (═O) group.

“Polymorphism” refers to when two or more clearly different phenotypes exist in the same population of a species where the occurrence of more than one form or morph. In order to be classified as such, morphs must occupy the same habitat at the same time and belong to a panmictic population (one with random mating).

“Protein binding” refers to the binding of a drug to proteins in blood plasma, tissue membranes, red blood cells and other components of blood.

“Protein shift” refers to determining a binding shift by comparing the EC₅₀ values determined in the absence and presence of human serum.

“Racemates” refers to a mixture of enantiomers. In an embodiment of the invention, the compounds of Formulas I or II or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N-moiety such as pyrazoles, imidazoles, benzimidazoles, triazoles, and tetrazoles.

The term ‘atropisomer’ refers to a stereoisomer resulting from an axis of asymmetry. This can result from restricted rotation about a single bond where the rotational barrier is high enough to allow differentiation of the isomeric species up to and including complete isolation of stable non-interconverting diastereomer or enantiomeric species. One skilled in the art will recognize that upon installing a nonsymmetrical R^{x} to core, the formation of atropisomers is possible. In addition, once a second chiral center is installed in a given molecule containing an atropisomer, the two chiral elements taken together can create diastereomeric and enantiomeric stereochemical species. Depending upon the substitution about the Cx axis, interconversion between the atropisomers may or may not be possible and may depend on temperature. In some instances, the atropisomers may interconvert rapidly at room temperature and not resolve under ambient conditions. Other situations may allow for resolution and isolation but interconversion can occur over a period of seconds to hours or even days or months such that optical purity is degraded measurably over time. Yet other species may be completely restricted from interconversion under ambient and/or elevated temperatures such that resolution and isolation is possible and yields stable species. When known, the resolved atropisomers were named using the helical nomenclature. For this designation, only the two ligands of highest priority in front and behind the axis are considered. When the turn priority from the front ligand 1 to the rear ligand 1 is clockwise, the configuration is P, if counterclockwise it is M.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” or “subject” refers to mammals and includes humans and non-human mammals.

Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Where specific compounds or generic formulas are drawn that have aromatic rings, such as aryl or heteroaryl rings, then it will be understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

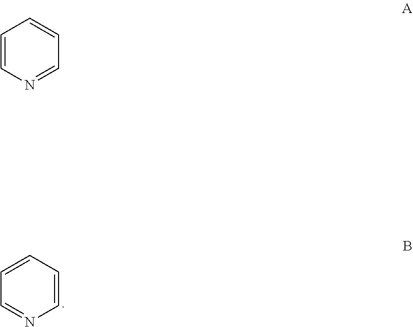 [SMILES: c1ccncc1 ; c1ccncc1]

The present invention includes compounds as well as their pharmaceutically acceptable salts. Accordingly, the word “or” in the context of “a compound or a pharmaceutically acceptable salt thereof” is understood to refer to either: 1) a compound alone or a compound and a pharmaceutically acceptable salt thereof (alternative), or 2) a compound and a pharmaceutically acceptable salt thereof (in combination).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—. In a term such as “—C(R^{x})₂”, it should be understood that the two R^{x} groups can be the same, or they can be different if R^{x} is defined as having more than one possible identity. In addition, certain substituents are drawn as —R^{x}R^{y}, where the “-” indicates a bond adjacent to the parent molecule and R^{y} being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

In one aspect, there is provided a compound of the formula (I):

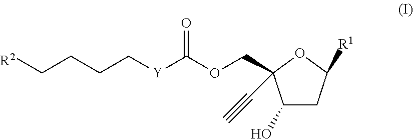

wherein:

R₁ is selected from the group consisting of:

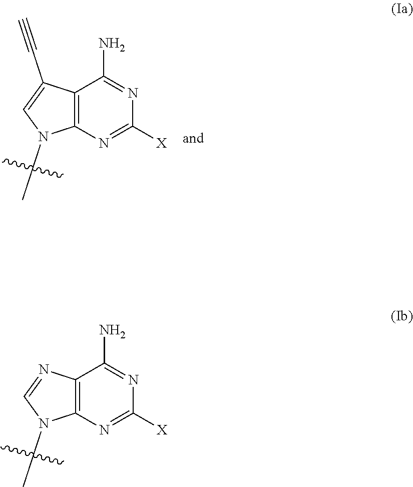

X is selected from the group consisting of NH₂, F and Cl;

Y is selected from the group consisting of a bond, (C₁-C₁₀) alkyl and CR³R³, wherein R³ and R^{3′} are independently selected from the group consisting of H, (C₁-C₆) alkyl, (C₁-C₆) haloalkyl, (C₂-C₁₀) alkenyl, (C₁-C₁₀) alkynyl and (C₃-C₁₄) cycloalkyl; and each of R³ and R^{3′} may be independently optionally substituted by (C₁-C₆) alkyl, Cl, F, oxo, or (C₁-C₆) alkoxy

R² is selected from the group consisting of H, (C₁-C₁₀) alkyl, (C₂-C₁₀) alkenyl, (C₂-C₁₀) alkynyl, (C₁-C₁₀) alkoxy and (C₁-C₁₀) haloalkyl; wherein each of R² may be optionally substituted by (C₁-C₆) alkyl, Cl, F, oxo, or (C₁-C₆) alkoxy

or a pharmaceutically acceptable salt thereof.

In one embodiment of the present invention, there is provided a compound of the formula (I), wherein Y is a bond and R² is H.

In one embodiment of the present invention, there is provided a compound of formula (I), wherein Y is (C₁-C₁₀) alkyl and R² is (C₁-C₁₀) alkyl.

In one embodiment of the present invention, there is provided a compound of formula (I), wherein Y is (C₁-C₁₀) alkyl and R² is (C₁-C₁₀) alkenyl.

In one embodiment of the present invention, there is provided a compound of formula (I), wherein R² is (C₁-C₁₀) alkyl.

In one embodiment of the present invention, there is provided a compound of formula (I) or a pharmaceutically acceptable salt thereof wherein X is F or C₁. In one embodiment, X is F. In another embodiment, X is C₁.

In one embodiment of the present invention, there is provided a compound of formula (I) or a pharmaceutically acceptable salt thereof wherein R³ and R⁴ are independently selected from (C₁-C₆) alkyl.

In one embodiment of the present invention, there is provided a compound of formula (I) or a pharmaceutically acceptable salt thereof, wherein Y is CR³R³, R^{3′} is (C₁-C₆) alkyl and R³ is H.

In one embodiment of the present invention, there is provided a compound of formula (I) or a pharmaceutically acceptable salt thereof, wherein R₁ is:

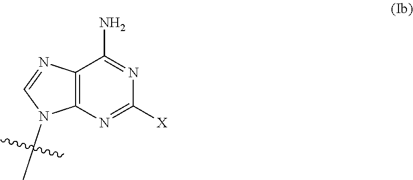

Preferably in this embodiment of the formula (Ib), X is F.

Preferably in this embodiment of the formula (Ib), wherein X is F, Y is a bond and R² is H.

Preferably in this embodiment of the formula (Ib), wherein X is F, Y is (C₁-C₁₀) alkyl and R² is (C₁-C₁₀) alkyl.

Preferably in this embodiment of the formula (Ib), wherein X is F, Y is (C₁-C₁₀) alkyl and R² is (C₁-C₁₀) alkenyl.

Preferably in this embodiment of the formula (Ib), X is F and Y and R² together are selected such that the alkyl chain:

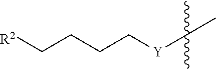

ranges from (C₁₀ to C₂₅) alkyl.

In another aspect, the invention relates to a compound of formula (II):

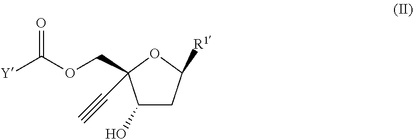

wherein:

R^{1′} is:

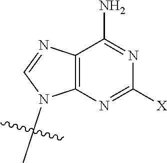

wherein:

X is selected from the group consisting of NH₂, F and Cl;

Y′ is selected from the group consisting of (C₁-C₇) alkyl, (C₁-C₆) haloalkyl, (C₂-C₆) alkenyl, (C₁-C₆) alkynyl, with the proviso that when Y′ is C₆ alkyl, it is present as a branched alkyl; and wherein Y′ may be optionally optionally substituted by Cl, F, oxo, alkoxy, or hydroxy;

or a pharmaceutically acceptable salt thereof.

In one embodiment of the present invention, there is provided a compound of formula (II), wherein Y′ is (C₁-C₆)alkyl, more preferably, (C₁-C₅) alkyl, most preferably, (C₁-C₄) alkyl.

In one embodiment of the present invention, there is provided a compound of formula (II), wherein when Y′ is C₇ alkyl, it cannot be a linear alkyl or a branched alkyl of the formula:

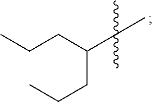 [SMILES: *C(CCC)CCC]

In one embodiment of the present invention, there is provided a compound of formula (II), wherein X is F or Cl. In one embodiment, X is F. In another embodiment, X is Cl.

In one embodiment of the present invention, there is provided a compound of formula (II), wherein Y′ is C₁ alkyl.

In one embodiment of the present invention, there is provided a compound of the formula (II), wherein Y′ is C₂ alkyl.

In one embodiment of the present invention, there is provided a compound of formula (II), wherein Y′ is C₃ alkyl.

In one embodiment of the present invention, there is provided a compound of formula (II), wherein Y′ is C₄ alkyl. In a preferred embodiment, C₄ alkyl is present as a linear alkyl. In a preferred embodiment, C₄ alkyl is present as a branched alkyl.

In another aspect, the invention relates to a compound of formula (III):

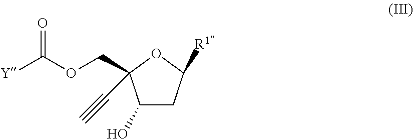

wherein:

R^{1″} is:

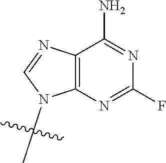 [SMILES: *n1cnc2c(N)nc(F)nc21]

and Y″ is selected from the group consisting of (C₁₀ to C₂₅) alkyl.

In another aspect of the present invention, the invention may encompass various individual compounds. As an example, such specific compounds may be selected from the group consisting of (Tables 1 and 2):

TABLE 1

| Parent Structure | Chemical Name |
|---|---|
| 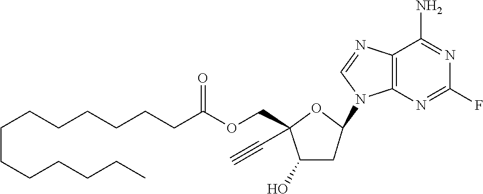 [SMILES: C#C[C@]1(COC(=O)CCCCCCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl tetradecanoate |
| 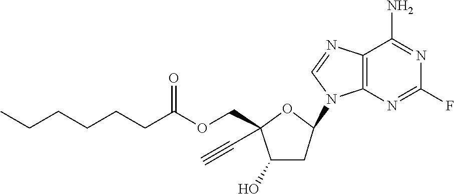 [SMILES: C#C[C@]1(COC(=O)CCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl heptanoate |
| 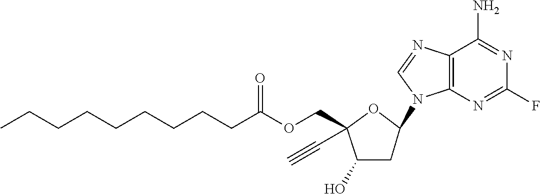 [SMILES: C#C[C@]1(COC(=O)CCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl decanoate |
| 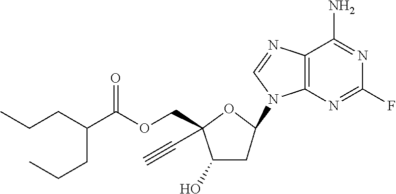 [SMILES: C#C[C@]1(COC(=O)C(CCC)CCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl 2-propylpentanoate |
| 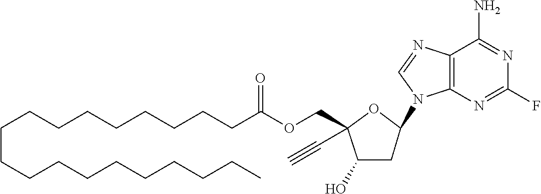 [SMILES: C#C[C@]1(COC(=O)CCCCCCCCCCCCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl icosanoate |
| 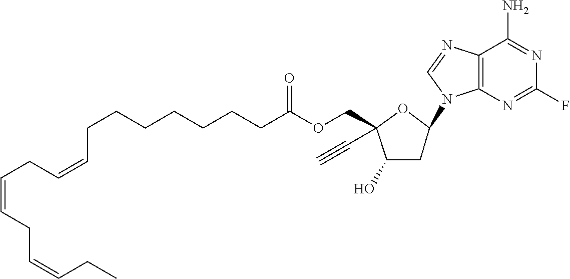 [SMILES: C#C[C@]1(COC(=O)CCCCCCC/C=C\C/C=C\C/C=C\CC)O[C@@H](n2cnc3c(N)nc(F)nc32)C[C@@H]1O] | (9Z,12Z,15Z)-((2R,3S,5R)-5-(6- amino-2-fluoro-9H-purin-9-yl)-2- ethynyl-3- hydroxytetrahydrofuran-2- yl)methyl octadeca-9,12,15- trienoate |

TABLE 2

| Parent Structure | Chemical Name |
|---|---|
| 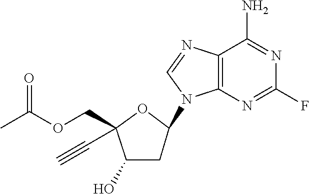 [SMILES: C#C[C@]1(COC(C)=O)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl acetate |
| 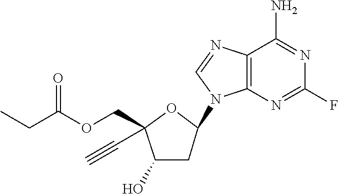 [SMILES: C#C[C@]1(COC(=O)CC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl propanoate |
| 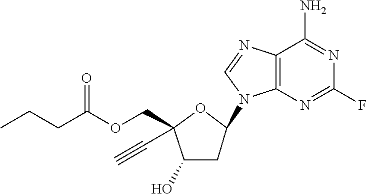 [SMILES: C#C[C@]1(COC(=O)CCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl butanoate |
| 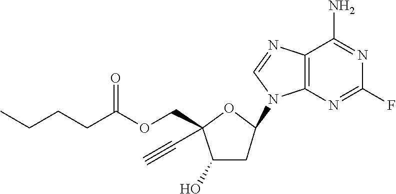 [SMILES: C#C[C@]1(COC(=O)CCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl pentanoate |
| 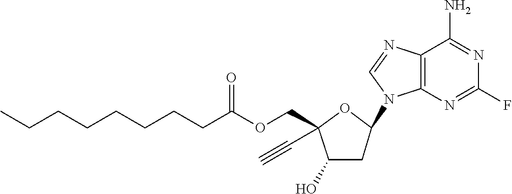 [SMILES: C#C[C@]1(COC(=O)CCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl nonanoate |
| 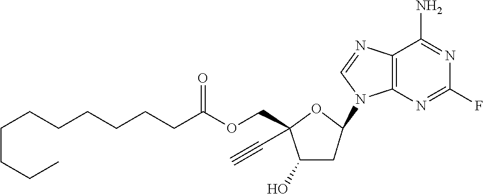 [SMILES: C#C[C@]1(COC(=O)CCCCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl undecanoate |
| 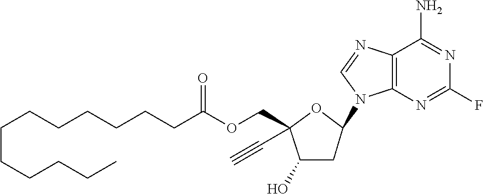 [SMILES: C#C[C@]1(COC(=O)CCCCCCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl tridecanoate |
| 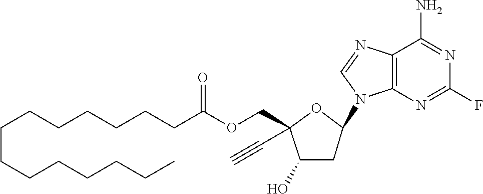 [SMILES: C#C[C@]1(COC(=O)CCCCCCCCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl pentadecanoate |
| 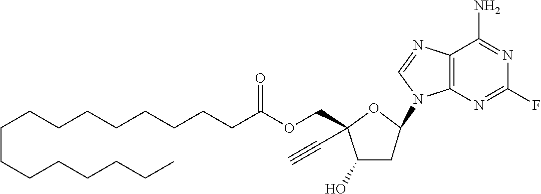 [SMILES: C#C[C@]1(COC(=O)CCCCCCCCCCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl heptadecanoate |
| 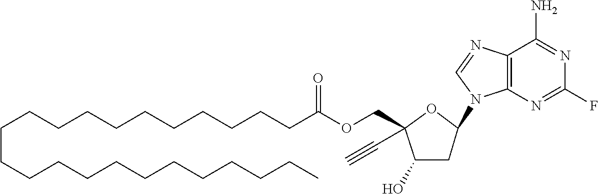 [SMILES: C#C[C@]1(COC(=O)CCCCCCCCCCCCCCCCCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl tetracosanoate |
| 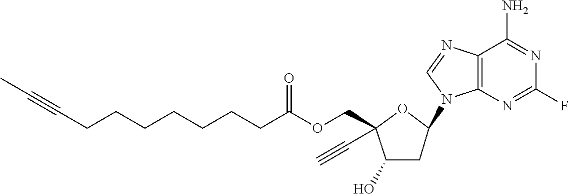 [SMILES: C#C[C@]1(COC(=O)CCCCCCCC#CC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl undec-9-ynoate |
| 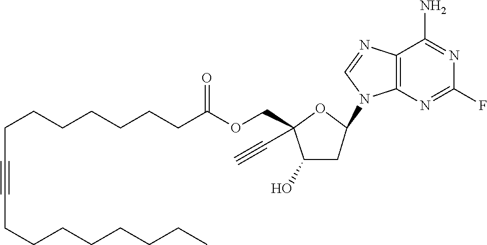 [SMILES: C#C[C@]1(COC(=O)CCCCCCCC#CCCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl octadec-9-ynoate |
| 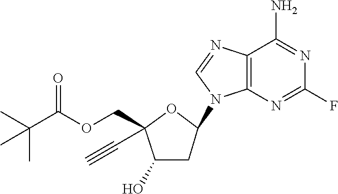 [SMILES: C#C[C@]1(COC(=O)C(C)(C)C)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 2,2-dimethylpropanoate |
| 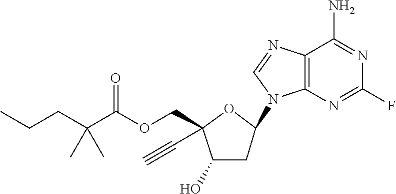 [SMILES: C#C[C@]1(COC(=O)C(C)(C)CCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 2,2-dimethylpentanoate |
| 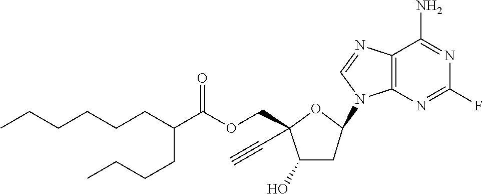 [SMILES: C#C[C@]1(COC(=O)C(CCCC)CCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 2-butyloctanoate |
| 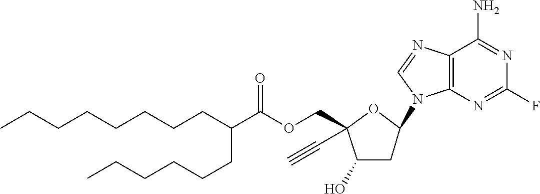 [SMILES: C#C[C@]1(COC(=O)C(CCCCCC)CCCCCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 2-hexyldecanoate |
| 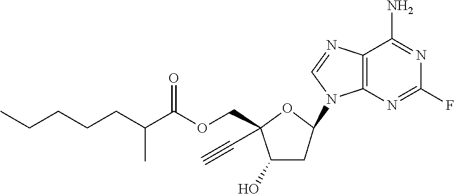 [SMILES: C#C[C@]1(COC(=O)C(C)CCCCC)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 2-methylheptanoate |
| 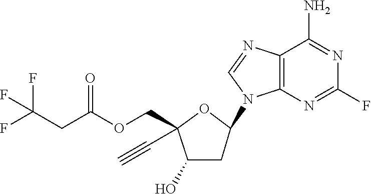 [SMILES: C#C[C@]1(COC(=O)CC(F)(F)F)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 3,3,3-trifluoropropanoate |
| 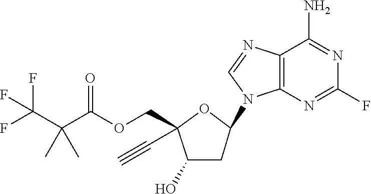 [SMILES: C#C[C@]1(COC(=O)C(C)(C)C(F)(F)F)O[C@@H](n2cnc3c(N)nc(F)nc32)C[C@@H]1O] | [(2R,3S,5R)-5-(6-amino-2-fluoro-9H- purin-9-yl)-2-ethynyl-3-hydroxyoxolan- 2-yl]methyl 3,3,3-trifluoro-2,2- dimethylpropanoate |

In one embodiment, the present invention encompasses each individual compound listed in the above Tables 1 and 2, or a pharmaceutically acceptable salt thereof.

Preferably, the invention provides a compound:

((2R,3S,5R)-5-(6-amino-2-fluoro-9H-purin-9-yl)-2-ethynyl-3-hydroxytetrahydrofuran-2-yl)methyl tetradecanoate

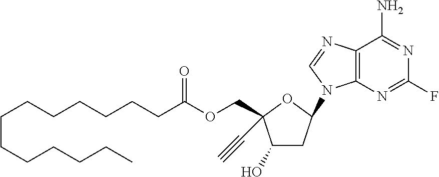 [SMILES: C#C[C@]1(COC(=O)CCCCCCCCCCCCC)O[C@@H](n2cnc3c(N)nc(F)nc32)C[C@@H]1O]

and a pharmaceutically acceptable salt thereof.

Preferably, the invention provides a compound:

((2R,3S,5R)-5-(6-amino-2-fluoro-9H-purin-9-yl)-2-ethynyl-3-hydroxytetrahydrofuran-2-yl)methyl icosanoate

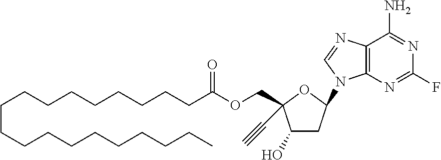 [SMILES: C#C[C@]1(COC(=O)CCCCCCCCCCCCCCCCCCC)O[C@@H](n2cnc3c(N)nc(F)nc32)C[C@@H]1O]

and a pharmaceutically acceptable salt thereof.

Preferably, the invention provides a compound:

((2R,3S,5R)-5-(6-amino-2-fluoro-9H-purin-9-yl)-2-ethynyl-3-hydroxytetrahydrofuran-2-yl)methyl heptadecanoate

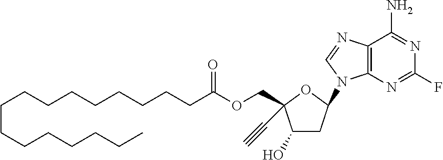 [SMILES: C#C[C@]1(COC(=O)CCCCCCCCCCCCCCCC)O[C@@H](n2cnc3c(N)nc(F)nc32)C[C@@H]1O]

and a pharmaceutically acceptable salt thereof.

Preferably, the invention provides a compound:

((2R,3S,5R)-5-(6-amino-2-fluoro-9H-purin-9-yl)-2-ethynyl-3-hydroxytetrahydrofuran-2-yl)methyl tetracosanoate

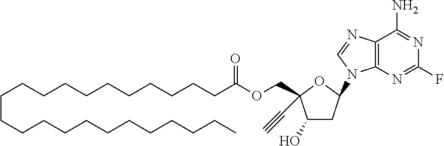 [SMILES: C#C[C@]1(COC(=O)CCCCCCCCCCCCCCCCCCCCCCC)O[C@@H](n2cnc3c(N)nc(F)nc32)C[C@@H]1O]

and a pharmaceutically acceptable salt thereof.

Preferably the invention provides a compound selected from the group consisting of:
a. ((2R,3S,5R)-5-(6-amino-2-fluoro-9H-purin-9-yl)-2-ethynyl-3-hydroxytetrahydrofuran-2-yl)methyl tetradecanoate

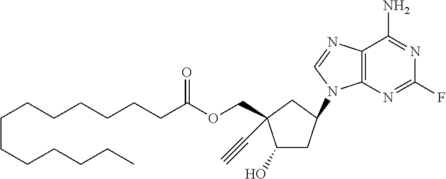 [SMILES: C#C[C@]1(COC(=O)CCCCCCCCCCCCC)C[C@@H](n2cnc3c(N)nc(F)nc32)C[C@@H]1O]

b. ((2R,3S,5R)-5-(6-amino-2-fluoro-9H-purin-9-yl)-2-ethynyl-3-hydroxytetrahydrofuran-2-yl)methyl icosanoate

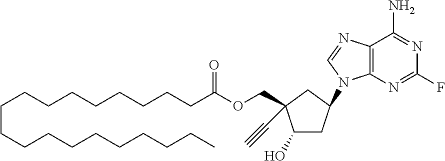 [SMILES: C#C[C@]1(COC(=O)CCCCCCCCCCCCCCCCCCC)C[C@@H](n2cnc3c(N)nc(F)nc32)C[C@@H]1O]

c. ((2R,3S,5R)-5-(6-amino-2-fluoro-9H-purin-9-yl)-2-ethynyl-3-hydroxytetrahydrofuran-2-yl)methyl heptadecanoate

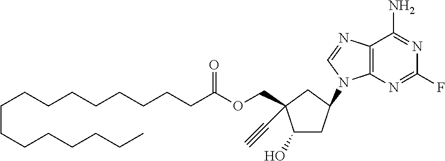 [SMILES: C#C[C@]1(COC(=O)CCCCCCCCCCCCCCCC)C[C@@H](n2cnc3c(N)nc(F)nc32)C[C@@H]1O]

d. ((2R,3S,5R)-5-(6-amino-2-fluoro-9H-purin-9-yl)-2-ethynyl-3-hydroxytetrahydrofuran-2-yl)methyl tetracosanoate

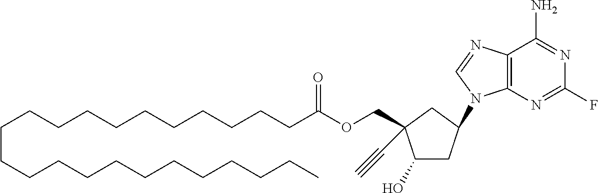 [SMILES: C#C[C@]1(COC(=O)CCCCCCCCCCCCCCCCCCCCCCC)C[C@@H](n2cnc3c(N)nc(F)nc32)C[C@@H]1O]

and pharmaceutically acceptable salts thereof.

In various embodiments, prodrugs of any of the compounds of formulas (I), (II) and (III) set forth herein are also within th scope of the present invention.

In accordance with one embodiment of the present invention, there is provided a pharmaceutical composition comprising a compound of Formulas (I), (II) and (III) or a pharmaceutically acceptable salt thereof, and a pharmaceutically acceptable excipient. In a further embodiment, the compound is present in amorphous form. In a further embodiment, the pharmaceutical composition is in a tablet form. In a further embodiment, the pharmaceutical composition is in parenteral form. In a further embodiment, the compound is present as a spray dried dispersion.

In accordance with one embodiment of the present invention, there is provided a method of treating an HIV infection in a subject comprising administering to the subject a compound of Formulas (I), (II) and (III) or a pharmaceutically acceptable salt thereof.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject a compound of Formulas (I), (II) and (III) or a pharmaceutically acceptable salt thereof.

In accordance with one embodiment of the present invention, there is provided the use of a compound of Formula (I), (II) and (III) in the manufacture of a medicament for treating an HIV infection.

In accordance with one embodiment of the present invention, there is provided the use of a compound of Formula (I), (II) and (III) in the manufacture of a medicament for preventing an HIV infection.

In accordance with one embodiment of the present invention, there is provided a compound according to Formula (I), (II) and (III) for use in treating an HIV infection.

In accordance with one embodiment of the present invention, there is provided a compound according to Formula (I), (II) and (III) for use in preventing an HIV infection.

In accordance with one embodiment of the present invention, there is provided a method of preventing an HIV infection in a subject at risk for developing an HIV infection, comprising administering to the subject a pharmaceutical composition as described herein.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formula (I), (II) and (III) wherein the compound or salt of the compound is used in the manufacture of a medicament for use in the treatment of an HIV infection in a human.

In another embodiment of the invention, there is provided a compound of Formula (I), (II) and (III) wherein the compound or salt of the compound is used in the manufacture of a medicament for use in the prevention of an HIV infection in a human.

In one embodiment, the pharmaceutical formulation containing a compound of Formula (I), (II) and (III) or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formula (I), (II) and (III) further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, vicriviroc (Sch-C), Sch-D, TAK779, maraviroc, TAK449, didanosine, tenofovir, lopinavir, and darunavir.

As such, the compounds of the present invention of Formulas (I), (II) and (III) and any other pharmaceutically active agent(s) may be administered together or separately and, when administered separately, administration may occur simultaneously or sequentially, in any order. The amounts of the compounds of Formula (I), (II) and (III) of the present invention and the other pharmaceutically active agent(s) and the relative timings of administration will be selected in order to achieve the desired combined therapeutic effect. The administration in combination of a compound of the present invention of Formula (I), (II) and (III) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formula (I), (II) and (III) or salts thereof and the other pharmaceutically active agent(s) and the relative timings of administration will be selected in order to achieve the desired combined therapeutic effect.

In addition, the compounds of the present invention of Formula (I), (II) and (III) may be used in combination with one or more other agents that may be useful in the prevention or treatment of HIV. Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068, BMS-626529, 5-Helix and similar agents;
Integrase inhibitors such as raltegravir, elvitegravir, dolutegravir, bictegravir, cabotegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

Further examples where the compounds of the present invention may be used in combination with one or more agents useful in the prevention or treatment of HIV are found in Table 3.

TABLE 3

| | | | |
|---|---|---|---|
| FDA | Brand | Generic | |
| Approval | Name | Name | Manufacturer |

Nucleoside Reverse Transcriptase Inhibitors (NRTIs)

| | | | |
|---|---|---|---|
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, ddl | Squibb |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, | GlaxoSmithKline |
| | | ABC | |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddl EC | Squibb |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + | GlaxoSmithKline |
| | | lamivudine | |
| 2004 | Truvada | emtricitabine + | Gilead Sciences |
| | | tenofovir disoproxil | |
| | | fumarate | |

Non-Nucleosides Reverse Transcriptase Inhibitors (NNRTIs)

| | | | |
|---|---|---|---|
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |

Protease Inhibitors (Pis)

| | | | |
|---|---|---|---|
| 1995 | lnvirase | saquinavir | Roche |
| | | mesylate, SQV | Pharmaceuticals |
| 1996 | Norvir | ritonavir, RTV | Abbott Laboratories |
| 1996 | Crixivan | indinavir, IDV | Merck |
| 1997 | Viracept | nelfinavir mesylate, | Pfizer |
| | | NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir + ritonavir | Abbott Laboratories |
| | | LPV/RTV | |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | Darunavir | Tibotec |
| | | | Therapeutics |

Fusion Inhibitors

| | | | |
|---|---|---|---|
| 2003 | Fuzeon | Enfuvirtide, T-20 | Roche |
| | | | Pharmaceuticals & |
| | | | Trimeris |

Entry Inhibitors

| | | | |
|---|---|---|---|
| 2007 | Selzentry | Maraviroc | Pfizer |

Integrase Inhibitors

| | | | |
|---|---|---|---|
| 2007 | Isentress | Raltegravir | Merck |
| 2013 | Tivicay | Dolutegravir | ViiV Healthcare |
| — | — | Cabotegravir | |

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City Calif. as a pharmacological enhancer.
SPI-452 is a compound being developed by Sequoia Pharmaceuticals of Gaithersburg, Md., as a pharmacological enhancer.

In one embodiment of the present invention, a compound of Formula (I), (II) and (III) is used in combination with ritonavir. In one embodiment, the combination is an oral fixed dose combination. In another embodiment, the compound of Formula (I), (II) and (III) is formulated as a long acting parenteral injection and ritonavir is formulated as an oral composition. In one embodiment, a kit containing the compound of Formula (I), (II) and (III) is formulated as a long acting parenteral injection and ritonavir formulated as an oral composition. In another embodiment, the compound of Formula (I), (II) and (III) is formulated as a long acting parenteral injection and ritonavir is formulated as an injectable composition. In one embodiment, a kit containing the compound of Formula (I), (II) and (III) is formulated as a long acting parenteral injection and ritonavir formulated as an injectable composition.

In another embodiment of the present invention, a compound of Formula (I), (II) and (III) is used in combination with GS-9350. In one embodiment, the combination is an oral fixed dose combination. In another embodiment, the compound of Formula (I), (II) and (III) is formulated as a long acting parenteral injection and GS-9350 is formulated as an oral composition. In one embodiment, there is provided a kit containing the compound of Formula (I), (II) and (III) is formulated as a long acting parenteral injection and GS-9350 formulated as an oral composition. In another embodiment, the compound of Formula (I), (II) and (III) is formulated as a long acting parenteral injection and GS-9350 is formulated as an injectable composition. In one embodiment, is a kit containing the compound of Formula (I), (II) and (III) is formulated as a long acting parenteral injection and GS-9350 formulated as an injectable composition.

In one embodiment of the present invention, a compound of Formula (I), (II) and (III) is used in combination with SPI-452. In one embodiment, the combination is an oral fixed dose combination. In another embodiment, the compound of Formula (I), (II) and (III) is formulated as a long acting parenteral injection and SPI-452 is formulated as an oral composition. In one embodiment, there is provided a kit containing the compound of Formula (I), (II) and (III) formulated as a long acting parenteral injection and SPI-452 formulated as an oral composition. In another embodiment, the compound of Formula (I), (II) and (III) is formulated as a long acting parenteral injection and SPI-452 is formulated as an injectable composition. In one embodiment, there is provided a kit containing the compound of Formula (I), (II) and (III) formulated as a long acting parenteral injection and SPI-452 formulated as an injectable composition.

In one embodiment of the present invention, a compound of Formula (I), (II) and (III) is used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III).

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III), wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III) further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III).

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III), wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III), further comprising administration of a therapeutically effective amount of one or more agents active against an HIV virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), (II) and (III) further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In further embodiments, the compound of the present invention of Formula (I), (II) and (III) or a pharmaceutically acceptable salt thereof, is selected from the group of compounds set forth in Tables 1 and 2 above.

The compounds of Tables 1 and 2 were synthesized according to the Synthetic Methods, General Schemes, and the Examples described below.

In another embodiment, there is provided a pharmaceutical composition comprising a pharmaceutically acceptable diluent and a therapeutically effective amount of a compound of Formula (I), (II) and (III) or a pharmaceutically acceptable salt thereof.

In certain embodiments, the compound(s) of the present invention, or a pharmaceutically acceptable salt thereof, is chosen from the compounds set forth in Tables 1 and 2. The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formula (I), (II) and (III) may be prepared. These pharmaceutically acceptable salts may be prepared in situ during the final isolation and purification of the compound, or by separately reacting the purified compound in its free acid or free base form with a suitable base or acid, respectively.

Illustrative pharmaceutically acceptable acid salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences, 17th ed., Mack Publishing Company, Easton, Pa., 1985, p. 1418, the disclosure of which is hereby incorporated by reference only with regards to the lists of suitable salts.

The compounds of Formula (I), (II) and (III) of the invention may exist in both unsolvated and solvated forms. The term ‘solvate’ comprises the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formula (I), (II) and (III) containing one or more asymmetric carbon atoms can exist as two or more stereoisomers. Where a compound of Formula (I), (II) and (III) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I), (II) and (III), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula (I), (II) and (III) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of Formula (I), (II) and (III) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), (II) and (III), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formula (I), (II) and (III) can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. Thus, certain derivatives of compounds of Formula (I), (II) and (III), which may have little or no pharmacological activity themselves can, when administered into or onto the body, be converted into compounds of Formula (I), (II) and (III) as ‘prodrugs’. One example of a compound that such prodrugs may encompass is 4′-ethylnyl-2-fluoro-2′-dooxyadenosine (EFdA) disclosed e.g., in U.S. Pat. No. 7,339,053. The compounds of the present invention may be administered as prodrugs. In one embodiment, the compounds of the invention are prodrugs of 4′-ethynyl-2-fluoro-2′-deoxyadenosine (EFdA) disclosed e.g., in U.S. Pat. No. 7,339,053, which is a nucleoside reverse transcriptase inhibitor of the formula:

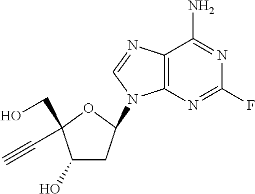 [SMILES: C#C[C@]1(CO)O[C@@H](n2cnc3c(N)nc(F)nc32)C[C@@H]1O]

The prodrugs are useful in that they are capable of modulating physicochemical properties, facilitating multiple dosing paradigms and improving pharmacokinetic and/or pharmacodynamic profiles of the active parent (EfdA). For example, the prodrugs may facilitate long-acting parenteral dosing modalities, and/or improvements in antiviral persistence profiles as compared to EFdA.

Administration of the chemical entities and combinations of entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. Examples of dosing include, without limitation, once every seven days for oral, once every eight weeks for intramuscular, or once every six months for subcutaneous.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition may comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In various embodiments, pharmaceutical compositions of the present invention encompass compounds of Formula (I), (II) and (III), salts thereof, and combinations of the above.

### Synthetic Methods

The methods of synthesis may employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein may or take place at atmospheric pressure, generally within a temperature range from −78° C. to 200° C. Further, except as employed in the Example or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLE AND GENERAL SYNTHESIS

The following examples and prophetic synthesis methods serve to more fully describe the manner of making and using the above-described invention. It is understood that this in no way serve to limit the true scope of the invention, but rather is presented for illustrative purposes. Unless otherwise specified, the following abbreviations have the following meanings. If an abbreviation is not defined, it has its generally accepted meaning.
aq.=Aqueous
μL=Microliters
μM=Micromolar
NMR=nuclear magnetic resonance
Boc=tert-butoxycarbonyl
Br=Broad
Cbz=Benzyloxycarbonyl
D=Doublet
Δ=chemical shift
° C.=degrees celcius
DCM=Dichloromethane
Dd=doublet of doublets DMAP=4-dimethylaminopyridine
DMEM=Dulbeco's Modified Eagle's Medium
DMF=N,N-dimethylformamide
DMSO=Dimethylsulfoxide
EtOAc=ethyl acetate
G=Gram
h or hr=Hours
HCV=hepatitus C virus
HPLC=high performance liquid chromatography
Hz=Hertz
IU=International Units
IC₅₀=inhibitory concentration at 50% inhibition
J=coupling constant (given in Hz unless otherwise indicated)
M=Multiplet
M=Molar
M+H⁺=parent mass spectrum peak plus H⁺
Mg=Milligram
Min=Minutes
mL=Milliliter
mM=Millimolar
Mmol=Millimole
MS=mass spectrum
Nm=Nanomolar
Ppm=parts per million
q.s.=sufficient amount
S=Singlet
RT=room temperature
sat.=Saturated
T=Triplet
   TBDPS=tert-butyldiphenylsilyl
   TEA=triethylamine
TFA=trifluoroacetic acid
   THF=tetrahydrofuran
   TMS=trimethyl silyl

Additionally, various compounds of the invention may be made, in one embodiment, by way of the general synthesis route set forth below:

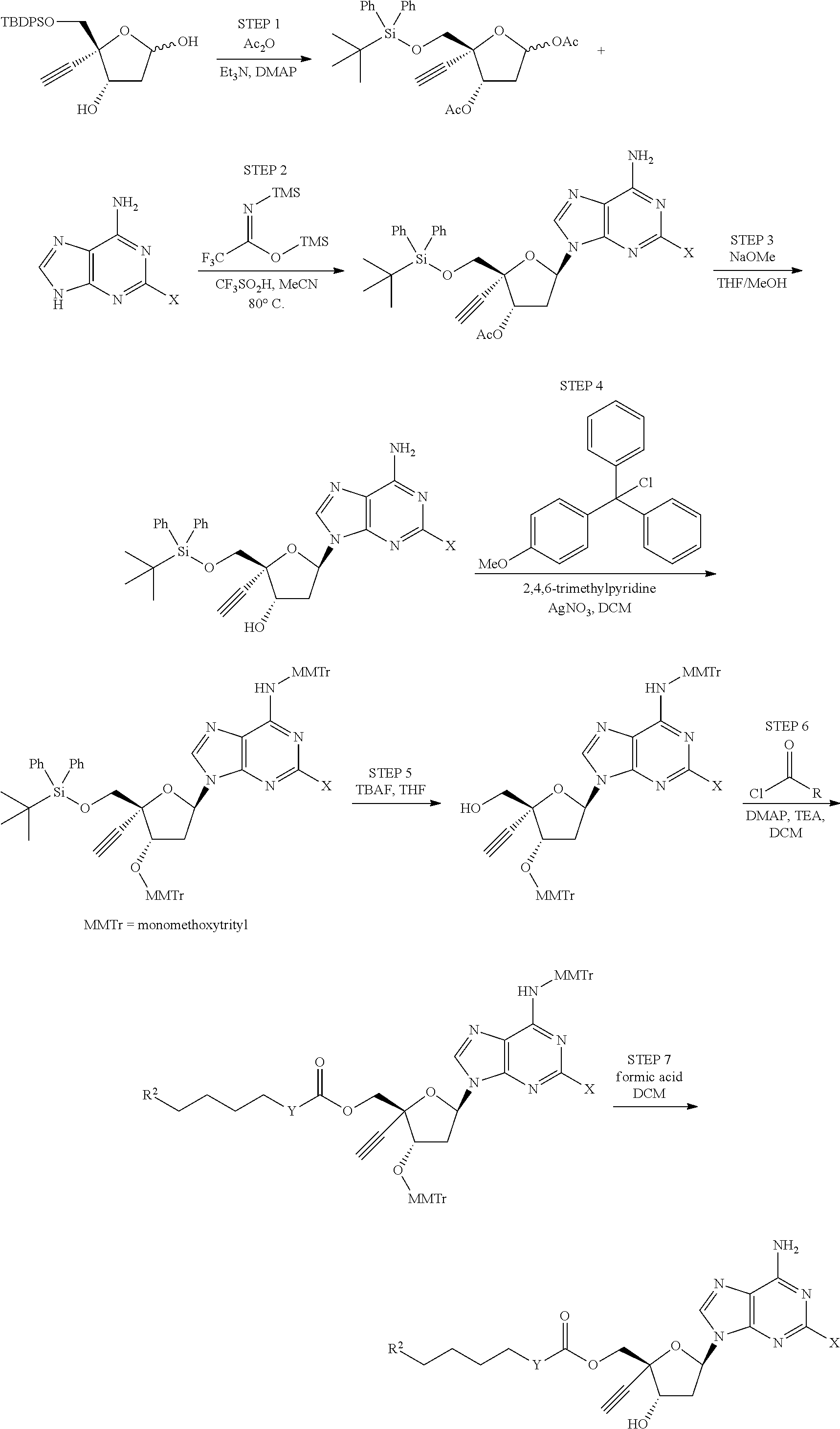

wherein X, Y and R² are defined herein.

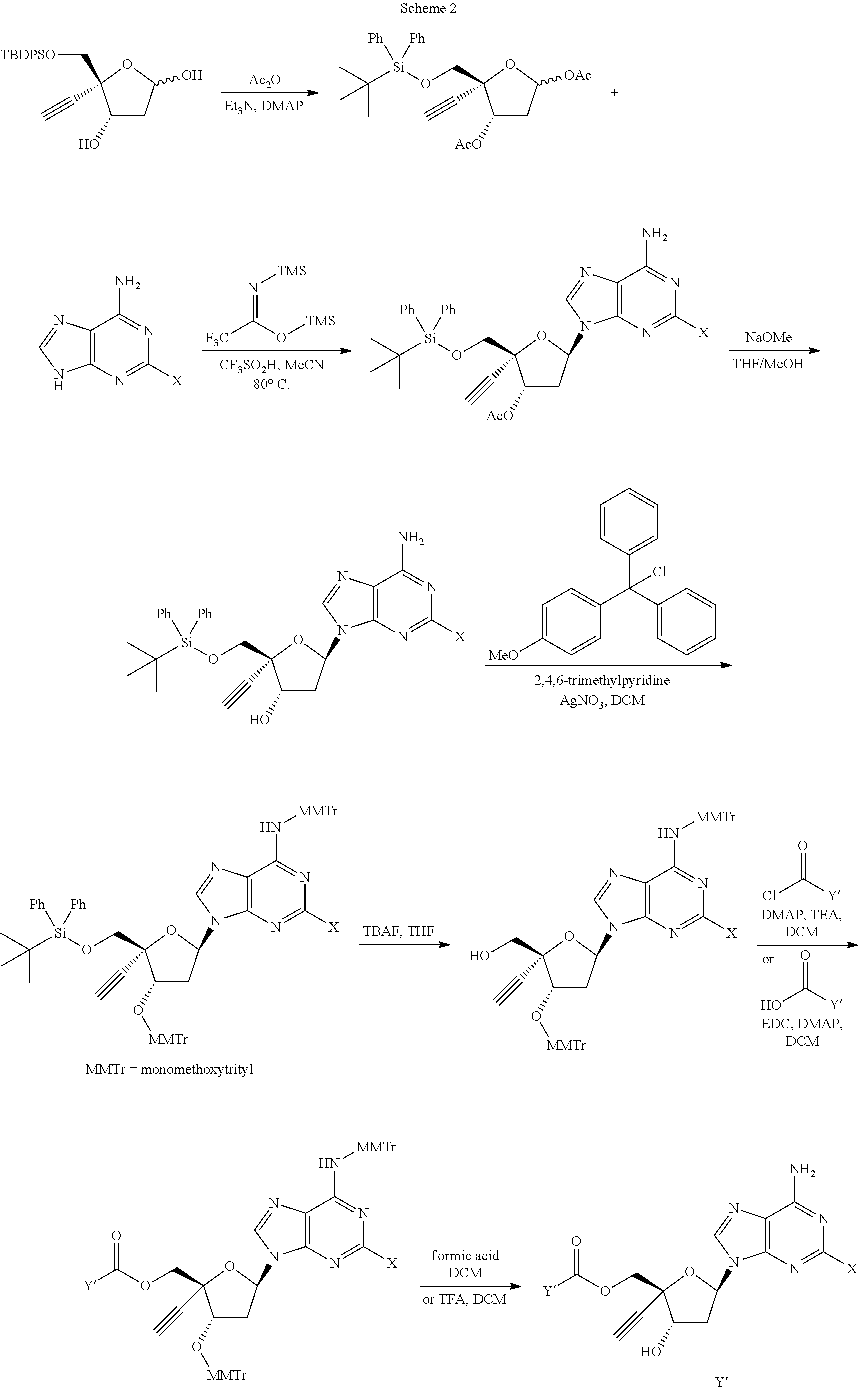

wherein Y′ is defined hereinabove

### Equipment Description

¹H NMR spectra were recorded on Varian or Bruker spectrometers. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Representative equipment and conditions for aquiring analytical low resolution LCMS are described below.

### Instrumentation:

Waters Acquity UPLC-MS system with SQ detectors

### MS Conditions:

Scan Mode: Alternating positive/negative electrospray

### Scan Range: 125-1200 amu

Scan Time: 150 msec
Interscan Delay: 50 msec

### LC Conditions:

The UPLC analysis was conducted on a Phenomenex Kinetex 1.7 um
2.1×50 mm XB-C18 column at 40° C.
0.2 uL of sample was injected using PLNO (partial loop with needle overfill) injection mode.
The gradient employed was:

### Mobile Phase A: Water+0.2% v/v Formic Acid

### Mobile Phase B: Acetonitrile+0.15% v/v Formic Acid

| Time | % A | % B | Flow Rate |
|---|---|---|---|
| 0.00 min | 95 | 5 | 1 ml/min |
| 1.1 min | 1 | 99 | 1 ml/min |
| 1.5 min | 1 | 99 | 1 ml/min |

UV detection provided by summed absorbance signal from 210 to 350 nm scanning at 40 Hz.

### Equipment Description

¹H NMR spectra were recorded on a Varian spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters (Acquity). The following conditions were employed described below.

### MS Conditions:

### Instrument: Waters SQD

### Serial Number: FO6SQD018N

### Scan Mode: Alternating Positive/Negative Electrospray

### Scan Range: 125-1200 amu

Scan Time: 150 msec
Interscan Delay: 50 msec

### LC Conditions:

The UPLC analysis was conducted on a Phenomenex Kinetex 1.7 um
2.1×50 mm XB-C18 column at 40° C.
0.2 uL of sample was injected using PLNO (partial loop with needle overfill) injection mode.
The gradient employed was:

### Mobile Phase A: Water+0.2% v/v Formic Acid

### Mobile Phase B: Acetonitrile+0.15% v/v Formic Acid

| Time | % A | % B | Flow Rate |
|---|---|---|---|
| 0.00 min | 95 | 5 | 1 ml/min |
| 1.1 min | 1 | 99 | 1 ml/min |
| 1.5 min | 1 | 99 | 1 ml/min |

UV detection provided by summed absorbance signal from 210 to 350 nm scanning at 40 Hz.

### Example 1: ((2R,3S,5R)-5-(6-amino-2-fluoro-9H-purin-9-yl)-2-ethynyl-3-hydroxytetrahydrofuran-2-yl)methyl tetradecanoate

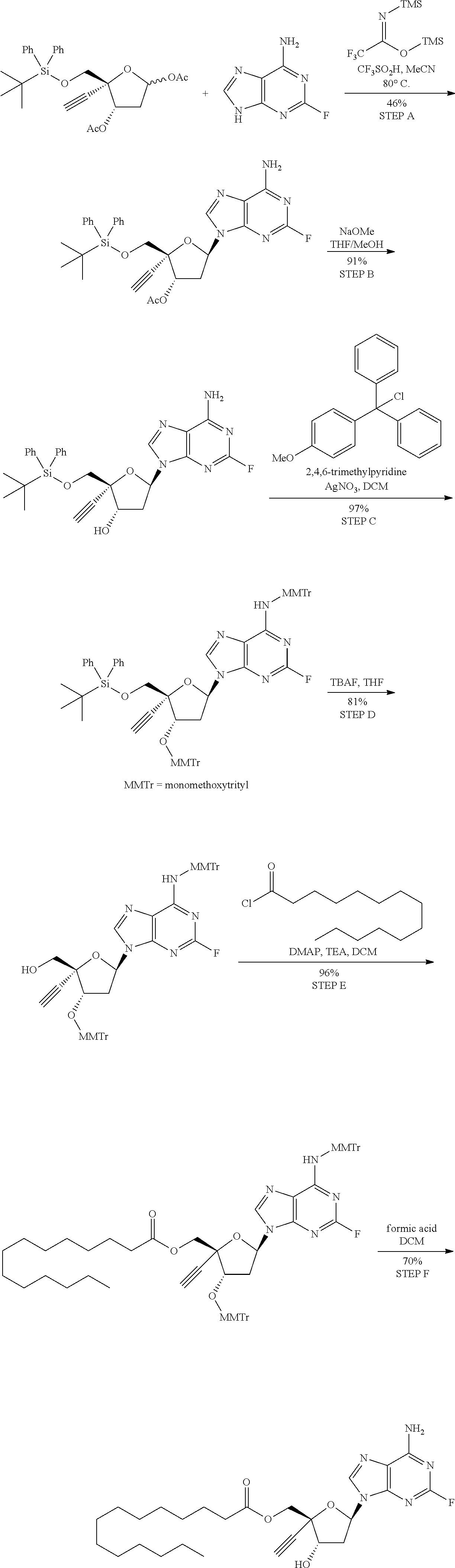

### Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl acetate. A suspension of

2-fluoro-9H-purin-6-amine (0.545 g, 3.56 mmol) in anhydrous MeCN (10 mL) in a screw-capped glass pressure vessel under a nitrogen atmosphere was treated with trimethylsilyl 2,2,2-trifluoro-N-(trimethylsilyl)acetimidate (1.89 ml, 7.12 mmol) and heated to 80° C. with stirring in an oil bath. After 45 minutes most of the solid had dissolved. The solution was treated with (4S,5R)-5-(((tert-butyldiphenylsilyl)oxy)methyl)-5-ethynyltetrahydrofuran-2,4-diyldiacetate (1.14 g, 2.37 mmol, prepared according to Org. Lett., Vol. 13, No. 19, 2011) dissolved in MeCN (9 mL) followed by freshly prepared 0.2M trifluoromethanesulfonic acid/MeCN (2.37 ml, 0.474 mmol) (prepared by dissolving 44 μL of triflic acid in 2.5 mL of MeCN). The temperature was maintained at 80° C. After 1.5 hour at 80° C. LCMS indicated complete reaction. The solution was cooled to RT, quenched by addition of 1M aqueous HCl (3 mL). After stirring the mixture briefly, it was partitioned between saturated aqueous NaHCO₃ and EtOAc and the phases separated. The aqueous phase was extracted with EtOAc (2×). The combined EtOAc solutions were dried over Na₂SO₄ and concentrated at reduced pressure to give a tan solid. This material was subjected to flash chromatography (silica gel, 0-100% EtOAc/DCM) and the higher R_{f} component isolated to afford the title compound (0.63 g, 46%) as a white solid. LCMS (ESI) m/z calcd for C₃₀H₃₂FN₅O₄Si: 573.2. Found: 574.4 (M+1)⁺. ¹H NMR (400 MHz, METHANOL-d₄) δ 8.10 (s, 1H), 7.59-7.67 (m, 4H), 7.26-7.45 (m, 6H), 6.39 (t, J=6.6 Hz, 1H), 5.91 (dd, J=7.0, 5.5 Hz, 1H), 3.97 (d, J=10.9 Hz, 1H), 3.86 (d, J=10.9 Hz, 1H), 3.05-3.18 (m, 2H), 2.64-2.74 (m, 1H), 2.14 (s, 3H), 0.97-1.04 (m, 9H).

### Step B: (2R,3S,5R)-5-(6-amino-2-fluoro-9H-purin-9-yl)-2-(((tert-butyldiphenylsilyl)oxy)methyl)-2-ethynyltetrahydrofuran-3-ol

To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (0.62 g, 1.08 mmol) in 1:1 THF/MeOH (4 mL) was added 25% NaOMe/MeOH (3 drops). The resulting solution was stirred at RT. After 30 minutes LCMS indicated complete reaction. The solution was treated with glacial AcOH (5 drops) and concentrated to dryness at reduced pressure. The residue was partitioned between 8:2 chloroform/iPrOH and half-saturated aqueous NaHCO₃ and the phases separated. The aqueous phase was extracted with two additional portions of 8:2 chloroform/iPrOH. The combined organic solutions were dried over Na₂SO₄ and concentrated to dryness at reduced pressure to afford the title compound (0.52 g, 91%) as a white solid. LCMS (ESI) m/z calcd for C₂₈H₃₀FN₅O₃Si: 531.2. Found: 532.3 (M+1)⁺. ¹H NMR (400 MHz, METHANOL-d₄) δ 8.17 (s, 1H), 7.53-7.66 (m, 4H), 7.22-7.45 (m, 6H), 6.32 (dd, J=7.8, 3.1 Hz, 1H), 5.01 (t, J=7.8 Hz, 1H), 3.87 (q, J=11.3 Hz, 2H), 3.05 (s, 1H), 2.90-2.99 (m, 1H), 2.63-2.72 (m, 1H), 0.94 (s, 9H).

### Step C: 9-(((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine

To a stirred suspension of (2R,3S,5R)-5-(6-amino-2-fluoro-9H-purin-9-yl)-2-(((tert-butyldiphenylsilyl)oxy)methyl)-2-ethynyltetrahydrofuran-3-ol (0.510 g, 0.959 mmol) in DCM (8 mL) was added silver nitrate (0.489 g, 2.88 mmol), 2,4,6-trimethylpyridine (0.766 ml, 5.76 mmol), and (chloro(4-methoxyphenyl)methylene)dibenzene (0.889 g, 2.88 mmol). The resulting orange suspension was stirred at RT. After 2 hours LCMS indicated complete reaction. The mixture was diluted with EtOAc and filtered through celite to remove solids. The filtrate was washed with 10% aqueous citric acid (2×), saturated aqueous NaHCO₃ (2×), dried over Na₂SO₄ and concentrated at reduced pressure to give a pale yellow foam. This material was subjected to flash chromatography (silica gel, 0-100% EtOAc/hexanes) to afford the title compound (1.00 g, 97%) as a white foam. LCMS (ESI) m/z calcd for C₆₈H₆₂FN₅O₅Si: 1075.5. Found: 1076.7 (M+1)⁺. ¹H NMR (400 MHz, CDCl₃) δ 7.12-7.62 (m, 35H), 6.98 (s, 1H), 6.74-6.82 (m, 4H), 6.22 (t, J=6.6 Hz, 1H), 4.75 (t, J=5.9 Hz, 1H), 3.93 (d, J=11.3 Hz, 1H), 3.86 (d, J=11.3 Hz, 1H), 3.77 (s, 3H), 3.75 (s, 3H), 2.77 (s, 1H), 1.71 (t, J=6.3 Hz, 2H), 0.87 (s, 9H).

### Step D: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol

To a stirred solution of 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine (0.99 g, 0.92 mmol) in THF (8 mL) was added 1M TBAF/THF (1.38 ml, 1.38 mmol) by dropwise addition. The resulting solution was stirred at RT. After 1 hour LCMS indicated complete reaction. The solution was treated with glacial AcOH (0.10 mL) and concentrated at reduced pressure. The residue was dissolved in MeOH/DCM and again concentrated to dryness. The residue was subjected to flash chromatography (silica gel, 0-100% EtOAc/hexanes) to afford the title compound (0.623 g, 81%) as a white solid. LCMS (ESI) m/z calcd for C₆₂H₄₄FN₆O₆: 837.3. Found: 838.6 (M+1)⁺. ¹H NMR (400 MHz, DMSO-d₆) δ 8.04 (s, 1H), 8.00 (s, 1H), 7.47-7.54 m, 4H), 7.12-7.38 (m, 20H), 6.79-6.88 (m, 4H), 6.04 (t, J=6.3 Hz, 1H), 5.15 (t, J=6.1 Hz, 1H), 4.47 (t, J=6.1 Hz, 1H), 3.84 (s, 1H), 3.69 (s, 3H), 3.67 (s, 3H), 3.49-3.57 (m, 1H), 3.38-3.47 (m, 1H), 1.63-1.72 (m, 1H), 1.49-1.58 (m, 1H).

### Step E: ((2R,3S,5R)-2-ethynyl-5-(2-fluoro-6-(((4-methoxyphenyl)diphenylmethyl)amino)-9H-purin-9-yl)-3-(4-methoxyphenyl)diphenylmethoxy)tetrahydrofuran-2-yl)methyl tetradecanoate

To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (25 mg, 0.030 mmol) and DMAP (1.8 mg, 0.015 mmol) in anhydrous DCM (1 mL) at 0° C. was added TEA (12.5 μl, 0.090 mmol) followed by a solution of tetradecanoyl chloride (8.1 mg, 0.033 mmol) in DCM (0.10 mL). The resulting solution was stirred at 0° C. for 10 minutes and then allowed to warm to RT. The reaction progress was monitored by TLC (silica gel, 8:2 EtOAc/hexanes). Two additional portions of all three reagents (same amounts as above) were added at t=30 min and t=1 hour. This afforded complete conversion of starting material to a new, higher Rf component. The solution was concentrated to dryness at reduced pressure and the residue subjected to flash chromatography (silica gel, 0-100% EtOAc/hexanes) to afford the title compound (30 mg, 96%). ¹H NMR (400 MHz, CDCl₃) δ 7.12-7.64 (m, 25H), 7.02 (br s, 1H), 6.79 (d, J=8.2 Hz, 4H), 6.02-6.13 (m, 1H), 4.60 (t, J=7.6 Hz, 1H), 4.31 (d, J=12.1 Hz, 1H), 4.05 (d, J=12.1 Hz, 1H), 3.78 (s, 3H), 3.72-3.74 (m, 3H), 2.82 (s, 1H), 2.15-2.27 (m, 1H), 1.92-2.12 (m, 2H), 0.58-1.80 (m, 26H).

### Step F: ((2R,3S,5R)-5-(6-amino-2-fluoro-9H-purin-9-0)-2-ethynyl-3-hydroxytetrahydrofuran-2-yl)methyl tetradecanoate

To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl tetradecanoate (29 mg, 0.028 mmol) in anhydrous DCM (1.2 mL) at 0° C. was added formic acid (106 μl, 2.77 mmol). The resulting solution was stirred at 0° C. for 10 minutes and then allowed to warm to RT. The reaction progress was monitored by HPLC. After 2 hours LCMS indicated a mixture of the desired product along with both mono-MMTr derivatives. The solution was treated with an additional 0.20 mL of formic acid. After another 1 hour LCMS indicated complete reaction. The solution was diluted with MeOH and then concentrated to dryness at reduced pressure. The residue was subjected to RP-HPLC purification (C18, 50-100% MeCN/water with 0.1% FA) to afford the title compound (9.7 mg, 70%) as a white solid. LCMS (ESI) m/z calcd for C₂₆H₃₈FN₅O₄: 503.3. Found: 504.4 (M+1)⁺. ¹H NMR (400 MHz, DMSO-d₆) δ 8.25 (s, 1H), 7.72-7.95 (m, 2H), 6.21 (dd, J=7.6, 3.7 Hz, 1H), 5.78 (d, J=5.1 Hz, 1H), 4.62-4.72 (m, 1H), 4.39 (d, J=11.9 Hz, 1H), 4.06 (d, J=11.9 Hz, 1H), 3.61 (s, 1H), 2.69-2.81 (m, 1H), 2.40-2.46 (m, 1H, overlapping DMSO peak), 2.05-2.27 (m, 2H), 1.04-1.44 (m, 22H), 0.82 (t, J=6.4 Hz, 3H).

### Example 2: ((2R,3S,5R)-5-(6-amino-2-fluoro-9H-purin-9-yl)-2-ethynyl-3-hydroxytetrahydrofuran-2-yl)methyl heptanoate

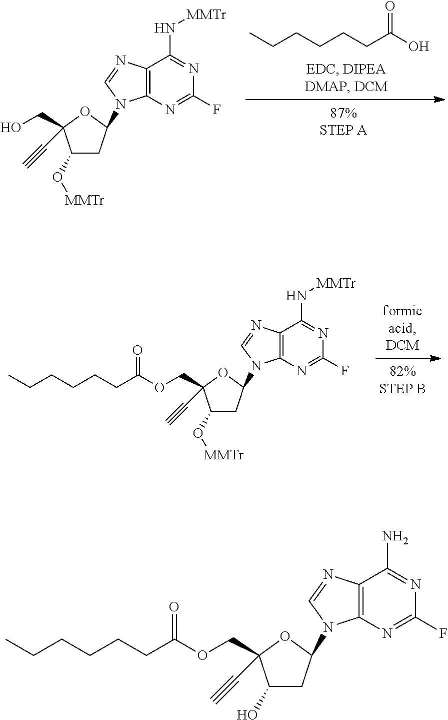

### Step A: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl heptanoate

To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (38 mg, 0.045 mmol), heptanoic acid (0.013 mL, 0.091 mmol) and DMAP (5.54 mg, 0.045 mmol) in DCM (0.6 mL) was added EDC (26.1 mg, 0.136 mmol), followed by DIPEA (0.040 mL, 0.227 mmol) at ambient temperature. LCMS after 45 minutes indicated complete reaction. The mixture was concentrated and then purified on silica gel (4 g column, 0-100% hexanes/EtOAc) to afford a colorless residue. LCMS (ESI) m/z calcd for C₅₉H₅₆FN₅O₆: 949.4. Found: 950.6 (M+1)⁺.

### Step B: ((2R,3S,5R)-5-(6-amino-2-fluoro-9H-purin-9-yl)-2-ethynyl-3-hydroxytetrahydrofuran-2-yl)methyl heptanoate

To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl heptanoate (37.5 mg, 0.039 mmol, 87% yield) in DCM (1.0 mL) was added formic acid (1.0 mL). The resulting orange solution was stirred for one hour and then concentrated. The residue was purified on silica gel (0-10% DCM/MeOH) to afford the title compound (12.9 mg, 70%) as a white residue. LCMS (ESI) m/z calcd for C₁₉H₂₄FN₅O₄: 405.2. Found: 406.3 (M+1)⁺. ¹H NMR (400 MHz, DMSO-d₆) δ 8.27 (s, 1H), 8.00-7.74 (m, 2H), 6.23 (dd, J=3.9, 8.2 Hz, 1H), 5.80 (d, J=5.5 Hz, 1H), 4.76-4.66 (m, 1H), 4.42 (d, J=11.7 Hz, 1H), 4.08 (d, J=11.7 Hz, 1H), 3.64 (s, 1H), 2.84-2.74 (m, 1H), 2.56-2.42 (m, 1H, overlapping DMSO peak), 2.28-2.05 (m, 2H), 1.49-1.32 (m, 2H), 1.27-1.10 (m, 6H), 0.81 (t, J=6.8 Hz, 3H).

### Example 3: ((2R,3S,5R)-5-(6-amino-2-fluoro-9H-purin-9-yl)-2-ethynyl-3-hydroxytetrahydrofuran-2-yl)methyl decanoate

The title compound was made in a similar manner as Example 2 except using decanoic acid in Step A. ¹H NMR (400 MHz, MeOH-d4) δ 8.16 (s, 1H), 6.30 (dd, J=3.3, 8.0 Hz, 1H), 4.96-4.81 (m, 1H, overlapping water peak), 4.48 (d, J=11.7 Hz, 1H), 4.25 (d, J=11.7 Hz, 1H), 3.17 (s, 1H), 2.96-2.87 (m, 1H), 2.72-2.62 (m, 1H), 2.31-2.14 (m, 2H), 1.55-1.40 (m, 2H), 1.35-1.16 (m, 12H), 0.89 (t, J=7.0 Hz, 3H). LCMS (ESI) m/z calcd for C₂₂H₃₀FN₅O₄: 447.2. Found: 448.3 (M+1)⁺.

### Example 4: ((2R,3S,5R)-5-(6-amino-2-fluoro-9H-purin-9-yl)-2-ethynyl-3-hydroxytetrahydrofuran-2-yl)methyl 2-propyl pentanoate

The title compound was made in a similar manner as Example 2 except using 2-propylpentanoic acid in Step A. ¹H NMR (400 MHz, DMSO-d₆) δ 8.28 (s, 1H), 7.99-7.73 (m, 2H), 6.25 (dd, J=4.1, 8.0 Hz, 1H), 5.81 (d, J=4.7 Hz, 1H), 4.73-4.63 (m, 1H), 4.39 (d, J=11.7 Hz, 1H), 4.08 (d, J=11.7 Hz, 1H), 3.63 (s, 1H), 2.88-2.80 (m, 1H), 2.54-2.42 (m, 1H, overlapping DMSO peak), 2.27-2.17 (m, 1H), 1.45-1.21 (m, 4H), 1.20-0.99 (m, 4H), 0.79-0.69 (m, 6H). LCMS (ESI) m/z calcd for C₂₀H₂₆FN₅O₄: 419.2. Found: 420.3 (M+1)⁺.

### Example 5: ((2R,3S,5R)-5-(6-amino-2-fluoro-9H-purin-9-yl)-2-ethynyl-3-hydroxytetrahydrofuran-2-yl)methyl icosanoate

The title compound was made in a similar manner as Example 2 except using icosanoic acid in Step A. ¹H NMR (400 MHz, DMSO-d₆) δ 8.26 (s, 1H), 7.97-7.74 (m, 2H), 6.23 (dd, J=3.9, 7.8 Hz, 1H), 5.79 (d, J=5.5 Hz, 1H), 4.75-4.66 (m, 1H), 4.41 (d, J=11.7 Hz, 1H), 4.08 (d, J=12.1 Hz, 1H), 3.63 (s, 1H), 2.82-2.74 (m, 1H), 2.54-2.41 (m, 1H, overlapping DMSO peak), 2.28-2.09 (m, 2H), 1.46-1.08 (m, 34H), 0.89-0.82 (m, 3H). LCMS (ESI) m/z calcd for C₃₂H₅₀FN₅O₄: 587.4. Found: 588.5 (M+1)⁺.

### Example 6: (9Z,12Z,15Z)-((2R,3S,5R)-5-(6-amino-2-fluoro-9H-purin-9-yl)-2-ethynyl-3-hydroxytetrahydrofuran-2-yl)methyl octadeca-9,12,15-trienoate

The title compound was made in a similar manner as Example 2 except using linolenic acid in Step A. ¹H NMR (400 MHz, MeOH-d₄) δ 8.15 (s, 1H), 6.30 (dd, J=3.5, 7.8 Hz, 1H), 5.43-5.22 (m, 6H), 4.95-4.81 (m, 1H, overlapping water peak), 4.48 (d, J=12.1 Hz, 1H), 4.26 (d, J=12.1 Hz, 1H), 3.17 (s, 1H), 2.95-2.87 (m, 1H), 2.83-2.77 (m, 4H), 2.71-2.62 (m, 1H), 2.31-2.14 (m, 2H), 2.12-2.00 (m, 4H), 1.56-1.41 (m, 2H), 1.37-1.15 (m, 8H), 0.96 (t, J=7.6 Hz, 3H). LCMS (ESI) m/z calcd for C₃₀H₄₀FN₅O₄: 553.3. Found: 554.4 (M+1)⁺.

### Example 7: ((2R,3S,5R)-5-(6-amino-2-fluoro-9H-purin-9-yl)-2-ethynyl-3-hydroxytetrahydrofuran-2-yl)methyl acetate

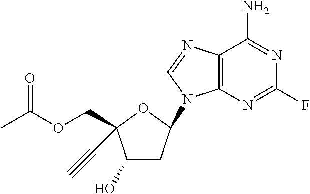 [SMILES: C#C[C@]1(COC(C)=O)O[C@@H](n2cnc3c(N)nc(F)nc32)C[C@@H]1O]

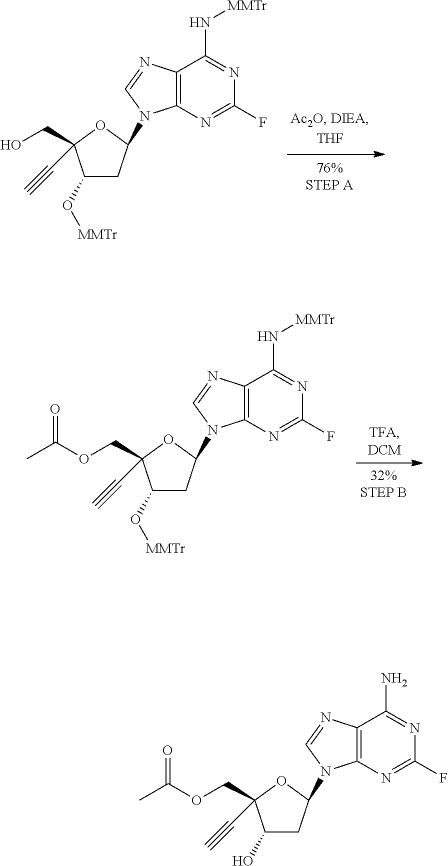

### Step A: ((2R,3S,5R)-2-ethynyl-5-(2-fluoro-6-(((4-methoxyphenyl)diphenylmethyl)amino)-9H-purin-9-14)-3-((4-methoxyphenyl)diphenylmethoxy)tetrahydrofuran-2-yl)methyl acetate

To a solution of ((2R,3S,5R)-2-ethynyl-5-(2-fluoro-6-(((4-methoxyphenyl)diphenylmethyl)amino)-9H-purin-9-yl)-3-(((4-methoxyphenyl)diphenylmethoxy)tetrahydrofuran-2-yl)methanol (150 mg, 0.179 mmol) and Ac₂O (0.034 mL, 0.358 mmol) in THF (2 mL) stirred under nitrogen at RT was added DIEA (0.063 mL, 0.358 mmol). The reaction mixture was stirred at RT for 2 hours. LCMS indicated complete reaction. The reaction mixture was concentrated under vacuum. The residue was subjected to preparative TLC (PE: EA=1:1) to give the desired product (92 mg, 76%) as a white solid. LCMS (ESI) m/z calcd for C₅₄H₄₆FN₅O₆: 879. Found: 880 (M+1)⁺. ¹H NMR (400 MHz, DMSO-d6) δ 8.09 (s, 1H), 7.99 (s, 1H), 7.51 (dt, J=8.2, 1.4 Hz, 4H), 7.33-7.16 (m, 20H), 6.89-6.83 (m, 4H), 6.15 (dd, J=7.9, 3.6 Hz, 1H), 4.72 (t, J=7.5 Hz, 1H), 4.10 (d, J=11.9 Hz, 1H), 3.95 (s, 1H), 3.71 (d, J=12.3 Hz, 6H), 3.65 (d, J=11.8 Hz, 1H), 2.10-2.06 (m, 1H), 2.00-1.95 (m, 1H), 1.91 (s, 3H).

### Step B: ((2R,3S,5R)-5-(6-amino-2-fluoro-9H-purin-9-yl)-2-ethynyl-3-hydroxytetrahydrofuran-2-yl)methyl acetate

A solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl acetate (92 mg, 0.105 mmol) in DCM (2 mL) and TFA (0.200 mL) was stirred under nitrogen at RT. The reaction mixture was stirred at RT for 2 hours. LCMS indicated complete reaction. The reaction was quenched with MeOH (5 mL) and concentrated under vacuum. The residue was purified by RP-HPLC (C₁₈, MeCN/water with 0.1% formic acid) to give the desired product (11.4 mg, 32%). LCMS (ESI) m/z calcd for C₁₄H₁₄FN₅O₄: 335. Found: 336 (M+1)+, ¹H NMR (400 MHz, Methanol-d₄) δ 8.16 (s, 1H), 6.31 (dd, J=7.9, 3.7 Hz, 1H), 4.87-4.85 (m, 1H), 4.46 (d, J=12.0 Hz, 1H), 4.27 (d, J=12.0 Hz, 1H), 3.17 (s, 1H), 2.91-2.85 (m, 1H), 2.69-2.62 (m, 1H), 1.97 (s, 3H).

### Example 8: ((2R,3S,5R)-5-(6-amino-2-fluoro-9H-purin-9-yl)-2-ethynyl-3-hydroxytetrahydrofuran-2-yl)methyl propionate

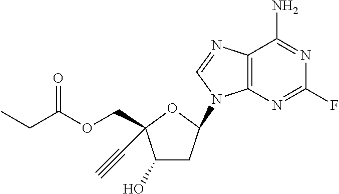 [SMILES: C#C[C@]1(COC(=O)CC)O[C@@H](n2cnc3c(N)nc(F)nc32)C[C@@H]1O]

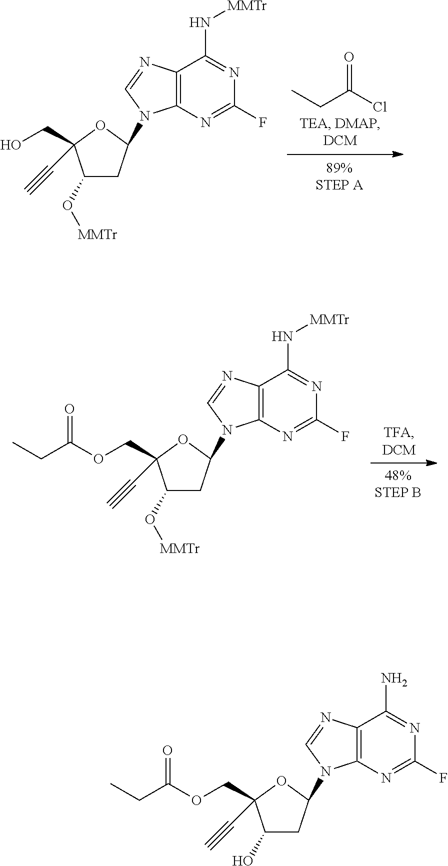

### Step A: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl propionate

((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (200 mg, 0.239 mmol) was dissolved in DCM (5 mL). TEA (0.100 mL, 0.716 mmol) and DMAP (14.6 mg, 0.119 mmol) were added, and then propionyl chloride (24.3 mg, 0.263 mmol) was added at 0° C. The reaction mixture was stirred at 0° C. overnight. LCMS indicated complete reaction. The solvent was removed under vacuum. The residue was subjected to preparative TLC (PE:EtOAc=2:1) to give the desired product (200 mg, 89%) as a yellow oil. LCMS (ESI) m/z calcd for C₅₅H₄₈FN₅O₆: 893. Found: 894 (M+1)⁺.

### Step B: ((2R,3S,5R)-5-(6-amino-2-fluoro-9H-purin-9-14)-2-ethynyl-3-hydroxytetrahydrofuran-2-yl)methyl propionate

((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl propionate (200 mg, 0.224 mmol) was dissolved in DCM (2 mL) and TFA (0.200 mL) and stirred for 0.5 h at RT. LCMS indicated complete reaction. The reaction mixture was diluted with MeOH (5 mL) and concentrated under vacuum. The residue was purified RP-HPLC (C18, MeCN/water with 0.1% formic acid) to give the product (37.4 mg, 48%). LCMS (ESI) m/z calcd for C₁₅H₁₆FN₅O₄:349. Found: 350 (M+1)⁺. ¹H NMR (400 MHz, Methanol-d₄): 8.16 (s, 1H), 6.32-6.29 (m, 1H), 4.88-4.85 (m, 1H), 4.48 (d, J=12.0 Hz, 1H), 4.26 (d, J=12.0 Hz, 1H), 3.17 (s, 1H), 2.90-2.87 (m, 1H), 2.67-2.64 (m, 1H), 2.34-2.21 (m, 2H), 1.02 (t, J=8.0 Hz, 3H).

### Example 9: ((2R,3S,5R)-5-(6-amino-2-fluoro-9H-purin-9-yl)-2-ethynyl-3-hydroxytetrahydrofuran-2-yl)methyl butyrate

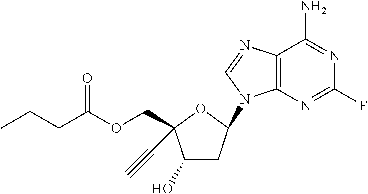 [SMILES: C#C[C@]1(COC(=O)CCC)O[C@@H](n2cnc3c(N)nc(F)nc32)C[C@@H]1O]

The title compound was prepared according to example 8, substituting butyryl chloride for propionyl chloride in Step A. LCMS (ESI) m/z calcd for C₁₆H₁₈FN₅O₄: 363. Found: 364 (M+1)⁺. ¹H NMR (400 MHz, Methanol-d₄) δ 8.15 (s, 1H), 6.30 (dd, J=8.0, 3.6 Hz, 1H), 4.89-4.88 (m, 1H), 4.48 (d, J=12.0 Hz, 1H), 4.24 (d, J=12.0 Hz, 1H), 3.17 (s, 1H), 2.97-2.94 (m, 1H), 2.67-2.63 (m, 1H), 2.30-2.12 (m, 2H), 1.53-1.50 (m, 2H), 0.86 (t, J=7.4 Hz, 3H).

### Example 10: ((2R,3S,5R)-5-(6-amino-2-fluoro-9H-purin-9-yl)-2-ethynyl-3-hydroxytetrahydrofuran-2-yl)methyl pentanoate

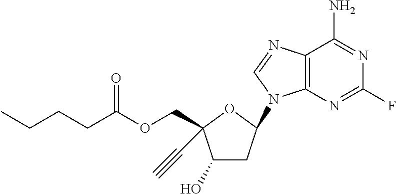 [SMILES: C#C[C@]1(COC(=O)CCCC)O[C@@H](n2cnc3c(N)nc(F)nc32)C[C@@H]1O]

The title compound was prepared according to example 8, substituting pentanoyl chloride for propionyl chloride in Step A. LCMS (ESI) m/z calcd for C₁₇H₂₀FN₅O₄: 377. Found: 378(M−1)⁺. ¹H NMR (400 MHz, DMSO-d₆) δ 8.28 (s, 1H), 7.87 (br, 2H), 6.24 (dd, J=8.0, 3.9 Hz, 1H), 5.80 (d, J=5.4 Hz, 1H), 4.74-4.69 (m, 1H), 4.43 (d, J=11.6 Hz, 1H), 4.09 (d, J=12.0 Hz, 1H), 3.64 (s, 1H), 2.83-2.78 (m, 1H), 2.49-2.44 (m, 1H), 2.27-2.08 (m, 2H), 1.45-1.33 (m, 2H), 1.24-1.14 (m, 2H), 0.80 (t, J=7.3 Hz, 3H).

### Example 11: ((2R,3S,5R)-5-(6-amino-2-fluoro-9H-purin-9-yl)-2-ethynyl-3-hydroxytetrahydrofuran-2-yl)methyl nonanoate

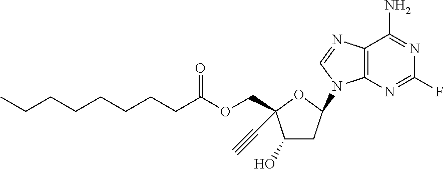 [SMILES: C#C[C@]1(COC(=O)CCCCCCCC)O[C@@H](n2cnc3c(N)nc(F)nc32)C[C@@H]1O]

The title compound was prepared according to example 8, substituting nonanoyl chloride for propionyl chloride in Step A. LCMS (ESI) m/z calcd for C₂₁H₂₈FN₅O₄:433. Found: 434 (M+1)⁺, ¹H NMR (400 MHz, Methanol-d₄) δ 8.15 (s, 1H), 6.30 (dd, J=8.0, 3.5 Hz, 1H), 4.88-4.86 (m, 1H), 4.47 (d, J=12.0 Hz, 1H), 4.25 (d, J=12.0 Hz, 1H), 3.17 (s, 1H), 2.90-2.88 (m, 1H), 2.69-2.64 (m, 1H), 2.24-2.20 (m, 2H), 1.48-1.46 (m, 2H), 1.30-1.22 (m, 10H), 0.88 (t, J=6.9 Hz, 3H).

### Example 12: ((2R,3S,5R)-5-(6-amino-2-fluoro-9H-purin-9-yl)-2-ethynyl-3-hydroxytetrahydrofuran-2-yl)methyl undecanoate

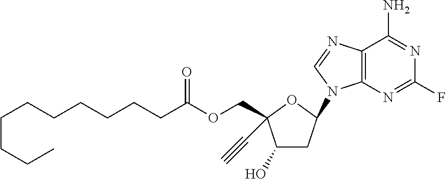 [SMILES: C#C[C@]1(COC(=O)CCCCCCCCCC)O[C@@H](n2cnc3c(N)nc(F)nc32)C[C@@H]1O]

The title compound was prepared according to example 8, substituting undecanoyl chloride for propionyl chloride in Step A. LCMS (ESI) m/z calcd for C₂₃H₃₂FN₅O₄: 461. Found: 462 (M+1)⁺. ¹H NMR (400 MHz, Chloroform-d) δ 7.92 (s, 1H), 6.33-6.30 (m, 1H), 6.03 (br, 2H), 4.77 (t, J=6.9 Hz, 1H), 4.45 (s, 2H), 2.99-2.93 (m, 1H), 2.71 (s, 1H), 2.69-2.64 (m, 1H), 2.41-2.28 (m, 2H), 1.60 (t, J=7.2 Hz, 2H), 1.32-1.19 (m, 14H), 0.88 (t, J=6.8 Hz, 3H).

### Example 13: ((2R,3S,5R)-5-(6-amino-2-fluoro-9H-purin-9-yl)-2-ethynyl-3-hydroxytetrahydrofuran-2-yl)methyl tridecanoate

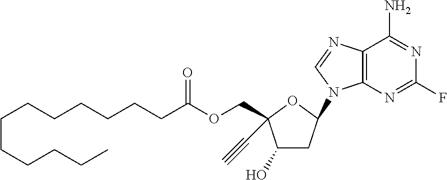 [SMILES: C#C[C@]1(COC(=O)CCCCCCCCCCCC)O[C@@H](n2cnc3c(N)nc(F)nc32)C[C@@H]1O]

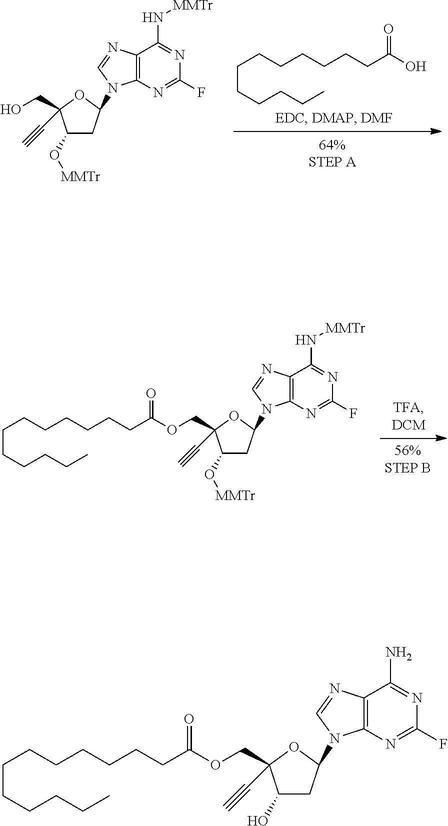

### Step A: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl tridecanoate

To a solution of tridecanoic acid (77 mg, 0.358 mmol) in DMF (5 mL) stirred at RT was added DMAP (109 mg, 0.895 mmol) and EDC (172 mg, 0.895 mmol). The reaction mixture was stirred at RT for 1 h. ((2R,3S,5R)-2-ethynyl-5-(2-fluoro-6-(((4-methoxyphenyl)diphenylmethyl)amino)-9H-purin-9-yl)-3-(((4-methoxyphenyl)diphenylmethoxy)tetrahydrofuran-2-yl)methanol (150 mg, 0.179 mmol) was added. The resulting mixture was stirred at RT overnight. LCMS indicated complete reaction. The reaction mixture was diluted with EtOAc and washed with brine, dried over Na₂SO₄ and concentrated under vacuum. The residue was subjected to preparative TLC (EtOAc:PE=1:1) to give the desired product (120 mg, 64%) as a white solid. LCMS (ESI) m/z calcd for C₆₅H₆₈FN₅O₆: 1033. Found: 1035 (M+1)⁺. ¹H NMR (400 MHz, Chloroform-d) δ 7.67 (s, 1H), 7.65-7.48 (m, 3H), 7.44-7.27 (m, 23H), 6.81 (dd, J=2.6, 9.0 Hz, 3H), 6.11 (dd, J=3.0, 7.9 Hz, 1H), 4.59 (t, J=7.7 Hz, 1H), 4.34 (d, J=12.2 Hz, 1H), 4.19-4.04 (m, 1H), 3.79-3.70 (m, 6H), 2.85 (s, 1H), 2.27-2.24 (m, 1H), 2.05-1.95 (m, 1H), 1.76-1.63 (m, 2H), 1.49-1.38 (m, 2H), 1.30-1.18 (m, 18H), 0.90 (t, J=6.8 Hz, 3H).

### Step B: ((2R,3S,5R)-5-(6-amino-2-fluoro-9H-purin-9-yl)-2-ethynyl-3-hydroxytetrahydrofuran-2-yl)methyl tridecanoate

A solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl tridecanoate (100 mg, 0.097 mmol) in DCM (5 mL) and TFA (0.5 mL) was stirred at room temperature for 3 h. LCMS indicated complete reaction. The reaction mixture was diluted with methanol (5 mL) and concentrated under vacuum. The residue was purified by RP-HPLC (C18, MeCN/water with 0.1% formic acid) to give the desired product (27 mg, 56%) as a white solid. LCMS (ESI) m/z calcd for C₂₅H₃₆FN₅O₄: 489. Found: 490 (M+1)⁺. ¹H NMR (400 MHz, Methanol-d₄) δ 8.15 (s, 1H), 6.30 (dd, J=3.6, 8.0 Hz, 1H), 4.97-4.85 (m, 1H), 4.70 (d, J=12.0 Hz, 1H), 4.25 (d, J=12.0 Hz, 1H), 3.16 (s, 1H), 2.93-2.87 (m, 1H), 2.70-2.62 (m, 1H), 2.30-2.14 (m, 2H), 1.54-1.16 (m, 20H), 0.91-0.87 (m, 3H).

### Example 14: ((2R,3S,5R)-5-(6-amino-2-fluoro-9H-purin-9-yl)-2-ethynyl-3-hydroxytetrahydrofuran-2-yl)methyl pentadecanoate

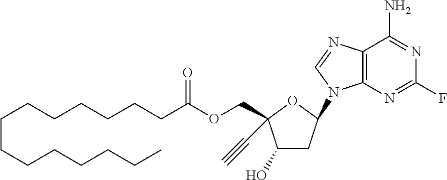 [SMILES: C#C[C@]1(COC(=O)CCCCCCCCCCCCCC)O[C@@H](n2cnc3c(N)nc(F)nc32)C[C@@H]1O]

The title compound was prepared according to example 13, substituting pentadecanoic acid for tridecanoic acid in Step A. LCMS (ESI) m/z calcd for C₂₇H₄₀FN₅O₄: 517. Found: 518 (M+1)⁺. ¹H NMR (400 MHz, DMSO-d₆) δ 8.26 (s, 1H), 7.85 (br, 2H), 6.24 (dd, J=3.6, 8.0 Hz, 1H), 5.79 (d, J=5.6 Hz, 1H), 4.73-4.67 (m, 1H), 4.41 (d, J=12.0 Hz, 1H), 4.08 (d, J=12.0 Hz, 1H), 3.63 (s, 1H), 2.81-2.75 (m, 1H), 2.48-2.43 (m, 1H), 2.27-2.10 (m, 2H), 1.40-1.16 (m, 24H), 0.87-0.83 (m, 3H).

### Example 15: ((2R,3S,5R)-5-(6-amino-2-fluoro-9H-purin-9-yl)-2-ethynyl-3-hydroxytetrahydrofuran-2-yl)methyl heptadecanoate

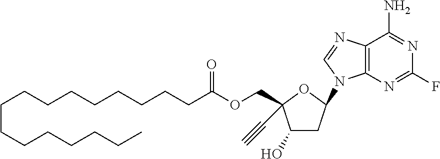 [SMILES: C#C[C@]1(COC(=O)CCCCCCCCCCCCCCCC)O[C@@H](n2cnc3c(N)nc(F)nc32)C[C@@H]1O]

The title compound was prepared according to example 13, substituting heptadecanoic acid for tridecanoic acid in Step A. LCMS (ESI) m/z calcd for C₂₉H₄₄FN₅O₄: 545. Found: 546 (M+1)⁺. ¹H NMR (400 MHz, Methanol-d₄) δ 8.15 (s, 1H), 6.31-6.29 (m, 1H), 4.89-4.87 (m, 1H), 4.47 (d, J=12.0 Hz, 1H), 4.25 (d, J=12.0 Hz, 1H), 3.17 (s, 1H), 2.94-2.88 (m, 1H), 2.70-2.62 (m, 1H), 2.30-2.15 (m, 2H), 1.53-1.47 (m, 2H), 1.34-1.24 (m, 26H), 0.91-0.88 (m, 3H).

### Example 16: ((2R,3S,5R)-5-(6-amino-2-fluoro-9H-purin-9-yl)-2-ethynyl-3-hydroxytetrahydrofuran-2-yl)methyl tetracosanoate

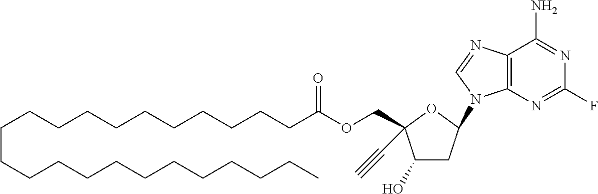 [SMILES: C#C[C@]1(COC(=O)CCCCCCCCCCCCCCCCCCCCCCC)O[C@@H](n2cnc3c(N)nc(F)nc32)C[C@@H]1O]

The title compound was prepared according to example 8, substituting tetracosanoyl chloride for propionyl chloride in Step A. LCMS (ESI) m/z calcd for C₇₆H₉₀FN₅O₆: 1188. Found: 1189 (M+1)⁺. ¹H NMR (400 MHz, Chloroform-d) δ 7.65 (s, 1H), 7.66-7.48 (m, 3H), 7.44-7.21 (m, 23H), 6.85-6.78 (m, 3H), 6.11 (dd, J=3.1, 7.9 Hz, 1H), 4.60 (t, J=7.7 Hz, 1H), 4.34 (d, J=12.3 Hz, 1H), 4.13-4.06 (m, 1H), 3.86-3.72 (m, 6H), 2.85 (s, 1H), 2.45-2.34 (m, 1H), 2.29-2.22 (m, 1H), 1.68-1.58 (m, 2H), 1.47-1.40 (m, 2H), 1.35-1.27 (m, 40H), 0.91 (t, J=6.7 Hz, 3H).

### Example 17: ((2R,3S,5R)-5-(6-amino-2-fluoro-9H-purin-9-yl)-2-ethynyl-3-hydroxytetrahydrofuran-2-yl)methyl undec-9-ynoate

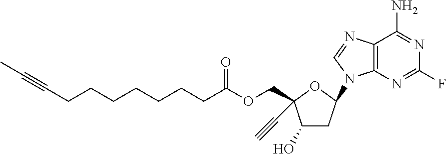 [SMILES: C#C[C@]1(COC(=O)CCCCCCCC#CC)O[C@@H](n2cnc3c(N)nc(F)nc32)C[C@@H]1O]

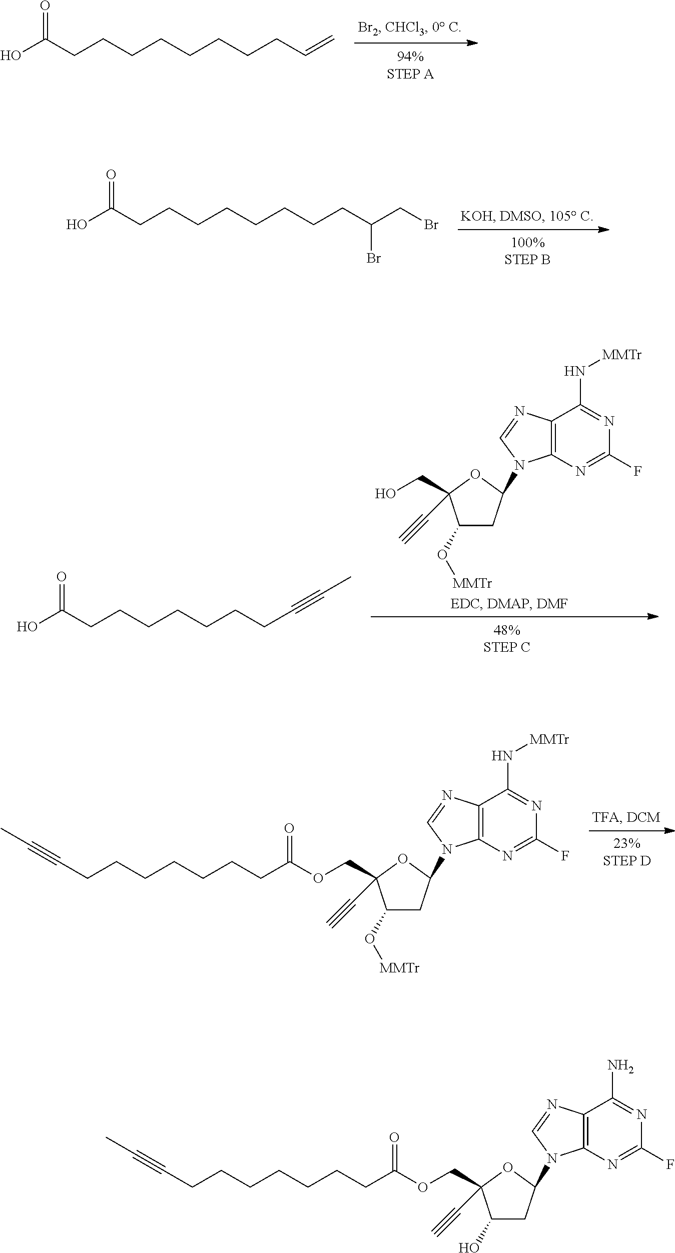

### Step A: 10,11-dibromoundecanoic acid

To a solution undec-10-enoic acid (2.00 g, 10.9 mmol) in CHCl₃ (7 mL) stirred at 0° C., a solution of bromine (0.590 mL, 11.4 mmol), in CHCl₃ (9 mL) was added below 5° C. and the mixture was stirred for 1 h at 0° C. LCMS indicated complete reaction. The reaction mixture was washed with sat. Na₂S₂O₃ (2×), sat. NaCl (1×). The organic phase was dried over Na₂SO₄ and concentrated under vacuum to give the desired product (3.6 g, 94%) as yellow oil (upon storing at 4° C., the oil solidified). LCMS (ESI) m/z calcd for C₁₁H₂₀Br₂O₂: 342. Found: 341 (M−1)⁻. ¹H NMR (400 MHz, Chloroform-d) δ 11.39 (br, 1H), 4.32-4.01 (m, 1H), 3.87 (dd, J=10.2, 4.4 Hz, 1H), 3.64 (t, J=10.0 Hz, 1H), 2.37 (t, J=7.5 Hz, 2H), 2.24-2.04 (m, 1H), 1.93-1.74 (m, 1H), 1.74-1.50 (m, 3H), 1.50-1.25 (m, 9H).

### Step B: undec-9-ynoic acid

To a solution of 10,11-dibromoundecanoic acid (500 mg, 1.45 mmol) in dimethyl sulfoxide (1.5 mL) stirred under nitrogen at 105° C. was added KOH (424 mg, 7.56 mmol). The reaction mixture was stirred at 105° C. for 3.5 hours. TLC showed that the reaction was complete. The mixture was diluted with MTBE and washed with 1M aqueous HCl. The organic phase was dried over Na₂SO₄ and concentrated under vacuum to give the desired product (310 mg, crude) as black oil. LCMS (ESI) m/z calcd for C₁₁H₁₈O₂: 182. Found: 181 (M−1)⁻. ¹H NMR (300 MHz, Chloroform-d) δ 2.38 (t, J=7.5 Hz, 3H), 2.14 (m, 2H), 1.80 (t, J=2.6 Hz, 2H), 1.51-1.44 (m, 2H), 1.36 (s, 8H).

### Step C: ((2R,3S,5R)-2-ethynyl-5-(2-fluoro-64(4-methoxyphenyl)diphenylmethyl)amino)-9H-purin-9-yl)-34(4-methoxyphenyl)diphenylmethoxy)tetrahydrofuran-2-yl)methyl undec-9-ynoate

To a solution of undec-9-ynoic acid (174 mg, 0.960 mmol), in DMF (3 mL) stirred at RT was added EDC (229 mg, 1.19 mmol), and DMAP (146 mg, 1.19 mmol). The reaction mixture was stirred at 30° C. for 2 hours. ((2R,3S,5R)-2-ethynyl-5-(2-fluoro-6-(((4-methoxyphenyl)diphenylmethyl)amino)-9H-purin-9-yl)-3-(((4-methoxyphenyl)diphenylmethoxy)tetrahydrofuran-2-yl)methanol (200 mg, 0.240 mmol) was added to the mixture. The mixture was stirred at 30° C. overnight. LCMS indicated complete reaction. Brine was added, and the mixture was extracted with EtOAc (4×). The organic phases were combined, dried over Na₂SO₄ and concentrated under vacuum. The residue was subjected to preparative TLC (PE/EtOAc=1:1) to give the desired product (130 mg, 48%) as a yellow solid. LCMS (ESI) m/z calcd for C₆₃H₆₀FN₅O₆: 1001. Found: 1002 (M+1)⁺. ¹H NMR (300 MHz, Chloroform-d) δ 7.86 (s, 1H), 7.68 (s, 1H), 7.54 (d, J=7.5 Hz, 3H), 7.35-7.30 (m, 16H), 7.26-7.24 (m, 2H), 6.90-6.77 (m, 7H), 6.16-6.08 (m, 1H), 4.82-4.73 (m, 1H), 4.63-4.55 (m, 1H), 4.34 (d, J=12.2 Hz, 1H), 3.83-3.80 (m, 6H), 3.76 (s, 1H), 3.00-2.93 (m, 1H), 2.69-2.60 (m, 1H), 2.39-2.35 (m, 2H), 2.17-2.11 (m, 2H), 1.83-1.79 (m, 3H), 1.69-1.63 (m, 4H), 1.51-1.46 (m, 4H), 1.34-1.32 (m, 2H).

### Step D: ((2R,3S5R)-5-(6-amino-2-fluoro-9H-purin-9-yl)-2-ethynyl-3-hydroxytetrahydrofuran-2-yl)methyl undec-9-ynoate

A solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl undec-9-ynoate (200 mg, 0.200 mmol) in DCM (2 mL) and TFA (0.200 mL) was stirred at RT for 30 minutes. LCMS indicated complete reaction. The mixture was diluted with methanol (5 ml) and concentrated under vacuum. The residue was purified by RP-HPLC (C₁₈, MeCN/water with 0.1% formic acid) to give the desired product (21.2 mg, 23%) as a white solid. LCMS (ESI) m/z calcd for C₂₃H₂₈FN₅O₄: 457. Found: 458(M−1)⁺. ¹H NMR (400 MHz, Methanol-d₄) δ 8.15 (s, 1H), 6.29 (dd, J=8.0, 3.5 Hz, 1H), 4.88-4.86 (m, 1H), 4.68 (d, J=12.0 Hz, 1H), 4.24 (d, J=12.0 Hz, 1H), 3.17 (s, 1H), 2.96-2.88 (m, 1H), 2.68-2.64 (m, 1H), 2.26-2.18 (m, 2H), 2.08-2.04 (m, 2H), 1.71 (t, J=2.6 Hz, 3H), 1.71-1.40 (m, 6H), 1.26-1.18 (m, 4H).

### Example 18: ((2R,3S,5R)-5-(6-amino-2-fluoro-9H-purin-9-yl)-2-ethynyl-3-hydroxytetrahydrofuran-2-yl)methyl octadec-9-ynoate

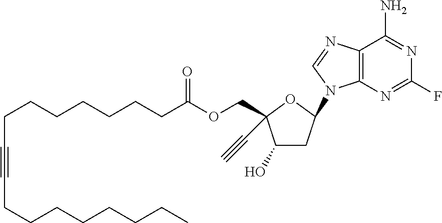 [SMILES: C#C[C@]1(COC(=O)CCCCCCCC#CCCCCCCCC)O[C@@H](n2cnc3c(N)nc(F)nc32)C[C@@H]1O]

The title compound was prepared according to example 13, substituting octadec-9-ynoic acid for tridecanoic acid in Step A. LCMS (ESI) m/z calcd for C₃₀H₄₂FN₅O₄:555. Found: 556 (M+1)⁺. ¹H NMR (400 MHz, CD3OD): 8.15 (s, 1H), 6.32-6.29 (m, 1H), 4.87-4.86 (m, 1H), 4.48 (d, J=12.0 Hz, 1H), 4.25 (d, J=12.0 Hz, 1H), 3.17 (s, 1H), 2.91-2.88 (m, 1H), 2.68-2.64 (m, 1H), 2.31-2.14 (m, 2H), 2.10-2.09 (m, 4H), 1.49-1.23 (m, 22H), 0.90-0.87 (m, 3H).

### Example 19: ((2R,3S,5R)-5-(6-amino-2-fluoro-9H-purin-9-yl)-2-ethynyl-3-hydroxytetrahydrofuran-2-yl)methyl pivalate

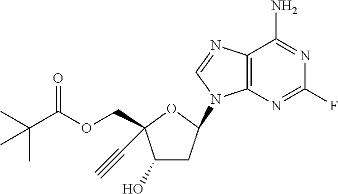 [SMILES: C#C[C@]1(COC(=O)C(C)(C)C)O[C@@H](n2cnc3c(N)nc(F)nc32)C[C@@H]1O]

The title compound was prepared according to example 2, substituting pivalic acid for heptanoic acid in Step A. LCMS (ESI) m/z calcd for C₁₇H₂₀FN₅O₄: 377.2. Found: 378.2 (M+1)⁺. ¹H NMR (400 MHz, DMSO-d6) δ=8.28 (s, 1H), 8.02-7.68 (m, 2H), 6.25 (dd, J=4.1, 8.0 Hz, 1H), 5.82 (d, J=5.5 Hz, 1H), 4.72-4.63 (m, 1H), 4.38 (d, J=11.7 Hz, 1H), 4.07 (d, J=12.1 Hz, 1H), 3.64 (s, 1H), 2.87-2.78 (m, 1H), 2.53-2.39 (m, 1H, overlapping DMSO peak), 1.06 (s, 9H).

### Example 20: ((2R,3S,5R)-5-(6-amino-2-fluoro-9H-purin-9-yl)-2-ethynyl-3-hydroxytetrahydrofuran-2-yl)methyl 2,2-dimethylpentanoate

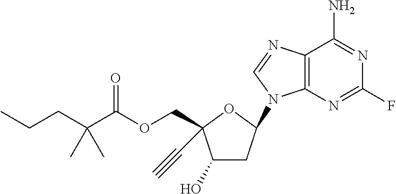 [SMILES: C#C[C@]1(COC(=O)C(C)(C)CCC)O[C@@H](n2cnc3c(N)nc(F)nc32)C[C@@H]1O]

The title compound was prepared according to example 2, substituting 2,2-dimethylpentanoic acid for heptanoic acid in Step A. LCMS (ESI) m/z calcd for C₁₉H₂₄FN₅O₄: 405.4. Found: 406.6 (M+1)⁺. ¹H NMR (400 MHz, Methanol-d₄) δ 8.14 (s, 1H), 6.32 (dd, J=3.8, 8.1 Hz, 1H), 4.88-4.82 (m, 1H), 4.39 (d, J=11.9 Hz, 1H), 4.24 (d, J=11.9 Hz, 1H), 3.15 (s, 1H), 3.05-2.97 (m, 1H), 2.71-2.96 (m, 1H), 1.46-1.30 (m, 2H), 1.18-1.06 (m, 8H), 0.81-0.71 (m, 3H).

### Example 21: ((2R,3S,5R)-5-(6-amino-2-fluoro-9H-purin-9-yl)-2-ethynyl-3-hydroxytetrahydrofuran-2-yl)methyl 2-butyloctanoate

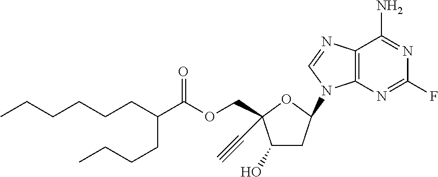 [SMILES: C#C[C@]1(COC(=O)C(CCCC)CCCCCC)O[C@@H](n2cnc3c(N)nc(F)nc32)C[C@@H]1O]

The title compound was prepared according to example 2, substituting 2-butyloctanoic acid for heptanoic acid in Step A. LCMS (ESI) m/z calcd for C₂₄H₃₄FN₅O₃₄: 475.6. Found: 476.4 (M+1)⁺. ¹H NMR (400 MHz, DMSO-d₆) δ 8.25 (s, 1H), 7.80 (br s, 2H), 6.25 (dd, J=4.1, 7.9 Hz, 1H), 5.76 (d, J=5.2 Hz, 1H), 4.74-4.63 (m, 1H), 4.37 (dd, J=2.7, 11.8 Hz, 1H), 4.11 (d, J=11.7 Hz, 1H), 3.60-3.58 (m, 1H), 2.88-2.76 (m, 1H), 2.56-2.41 (m, 1H, overlapping DMSO peak), 2.27-2.13 (m, 1H), 1.50-0.97 (m, 16H), 0.86-0.69 (m, 6H).

### Example 22: ((2R,3S,5R)-5-(6-amino-2-fluoro-9H-purin-9-yl)-2-ethynyl-3-hydroxytetrahydrofuran-2-yl)methyl 2-hexyldecanoate

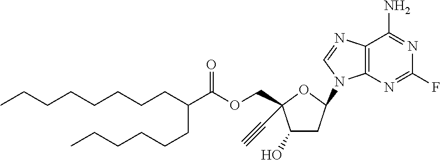 [SMILES: C#C[C@]1(COC(=O)C(CCCCCC)CCCCCCCC)O[C@@H](n2cnc3c(N)nc(F)nc32)C[C@@H]1O]

The title compound was prepared according to example 2, substituting 2-hexyldecanoic acid for heptanoic acid in Step A. LCMS (ESI) m/z calcd for C₂₈H₄₂FN₅O₄: 531.7. Found: 532.5 (M+1)⁺. ¹H NMR (400 MHz, DMSO-d₆) δ 8.24 (s, 1H), 7.79 (br s, 2H), 6.24 (dd, J=4.2, 8.0 Hz, 1H), 5.76 (d, J=5.2 Hz, 1H), 4.72-4.61 (m, 1H), 4.36 (dd, J=1.5, 11.8 Hz, 1H), 4.12 (dd, J=1.2, 11.7 Hz, 1H), 3.58-3.57 (m, 1H), 2.86-2.73 (m, 1H), 2.57-2.43 (m, 1H, overlapping DMSO peak), 2.27-2.14 (m, 1H), 1.48-0.97 (m, 24H), 0.88-0.77 (m, 6H).

### Example 23: ((2R,3S,5R)-5-(6-amino-2-fluoro-9H-purin-9-yl)-2-ethynyl-3-hydroxytetrahydrofuran-2-yl)methyl 2-methylheptanoate

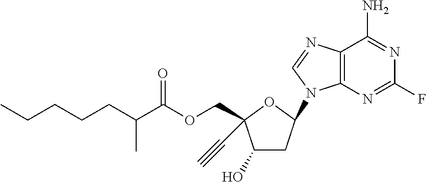 [SMILES: C#C[C@]1(COC(=O)C(C)CCCCC)O[C@@H](n2cnc3c(N)nc(F)nc32)C[C@@H]1O]

The title compound was prepared according to example 2, substituting 2-methylheptanoic acid for heptanoic acid in Step A. LCMS (ESI) m/z calcd for C₂₀H₂₆FN₅O₄: 419.4. Found: 420.3 (M+1)⁺. ¹H NMR (400 MHz, DMSO-d₆) δ 8.27 (d, J=2.4 Hz, 1H), 7.82 (br s, 2H), 6.25 (dd, J=4.1, 7.9 Hz, 1H), 5.78 (dd, J=1.2, 5.5 Hz, 1H), 4.77-4.61 (m, 1H), 4.41 (dd, J=4.9, 11.8 Hz, 1H), 4.10 (dd, J=1.5, 11.8 Hz, 1H), 3.62-3.61 (m, 1H), 2.87-2.77 (m, 1H), 2.54-2.44 (m, 1H, overlapping DMSO peak), 2.38-2.24 (m, 1H), 1.50-1.39 (m, 1H), 1.31-1.06 (m, 7H), 1.04-0.92 (m, 3H), 0.86-0.71 (m, 3H).

### Example 24: ((2R,3S,5R)-5-(6-amino-2-fluoro-9H-purin-9-yl)-2-ethynyl-3-hydroxytetrahydrofuran-2-yl)methyl 3,3,3-trifluoropropanoate

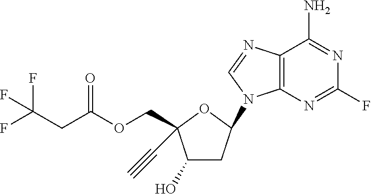 [SMILES: C#C[C@]1(COC(=O)CC(F)(F)F)O[C@@H](n2cnc3c(N)nc(F)nc32)C[C@@H]1O]

The title compound was prepared according to example 8, substituting 3,3,3-trifluoropropanoyl chloride for propionyl chloride in Step A. LCMS (ESI) m/z calcd for C₁₅H₁₃F₄N₅O₄:403. Found: 404 (M+1)⁺. ¹H NMR (300 MHz, Methanol-d₄): 8.14 (s, 1H), 6.33-6.30 (m, 1H), 4.87-4.85 (m, 1H), 4.58 (d, J=12.0 Hz, 1H), 4.35 (d, J=12.0 Hz, 1H), 3.38-3.30 (m, 2H), 3.20 (s, 1H), 2.90-2.87 (m, 1H), 2.68-2.63 (m, 1H).

### Example 25: ((2R,3S,5R)-5-(6-amino-2-fluoro-9H-purin-9-yl)-2-ethynyl-3-hydroxytetrahydrofuran-2-yl)methyl 3,3,3-trifluoro-2,2-dimethylpropanoate

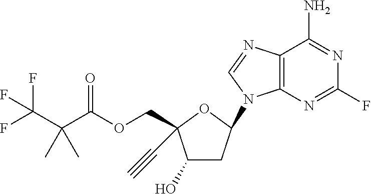 [SMILES: C#C[C@]1(COC(=O)C(C)(C)C(F)(F)F)O[C@@H](n2cnc3c(N)nc(F)nc32)C[C@@H]1O]

The title compound was prepared according to example 13, substituting 3,3,3-trifluoro-2,2-dimethylpropanoic acid for tridecanoic acid in Step A. LCMS (ESI) m/z calcd for C₁₇H₁₇F₄N₅O₄:431. Found: 432 (M+1)⁺. ¹H NMR (400 MHz, Methanol-d₄): 8.14 (s, 1H), 6.34-6.31 (m, 1H), 4.82 (t, J=7.6 Hz, 1H), 4.54 (d, J=12.0 Hz, 1H), 4.35 (d, J=12.0 Hz, 1H), 3.19 (s, 1H), 3.99-2.95 (m, 1H), 2.69-2.62 (m, 1H), 1.35 (d, J=4.0 Hz, 6H).

### Anti-HIV Activity

### PSV Assay

A pseudotyped virus assay (PSV) was used to assess the potency of the compounds. Replication defective virus was produced by co-transfection of a plasmid containing an NL4-3 provirus [containing a mutation in the envelope open reading frame (ORF) and a luciferase reporter gene replacing the nef ORF] and a CMV-promoter expression plasmid containing an ORF for various HIV gp160 envelope clones. The harvested virus was stored at −80 C in small aliquots and the titer of the virus measured to produce a robust signal for antiviral assays.

The PSV assay was performed by using U373 cells stably transformed to express human CD4, the primary receptor for HIV entry and either human CXCR4 or human CCR5 which are the co-receptors required for HIV entry as target cells for infection. Molecules of interest (including, but not limited to small molecule inhibitors of HIV, neutralizing antibodies of HIV, antibody-drug conjugate inhibitors of HIV, peptide inhibitors of HIV, and various controls) are capable of being diluted into tissue culture media and diluted via serial dilution to create a dose range of concentrations, and this was carried out for Example 1. This dose-range was applied to U373 cells and the pre-made pseudotyped virus added. The amount of luciferase signal produced after 3 days of culture was used to reflect the level of pseudotyped virus infection. An IC₅₀, or the concentration of inhibitor required (Example 1) to reduce PSV infection by 50% from the infection containing no inhibitor was calculated. Assays to measure cytotoxity were performed in parallel to ensure the antiviral activity observed for inhibitors was distinguishable from reduced target cell viability. IC₅₀ values were determined from a 10 point dose response curve using 3-4-fold serial dilution for each compound, which spans a concentration range>1000 fold.

These values are plotted against the molar compound concentrations using the standard four parameter logistic equation:

y=((Vmax*x\hat{ }n)/(K\hat{ }n+x\hat{ }n))+Y2
where:
Y2=minimum y n=slope factor
Vmax=maximum y x=compound concentration [M]
K=EC₅₀

The resulting data is shown in Tables 4 and 5. The IC₅₀ values shown in Table 5 for Examples 1-6 are believed to vary slightly from the analogous values in Table 4 due to a greater number of data replicates used to compute the mean values illustrated in Table 5.

TABLE 4

| | WT IC₅₀ | M184V |
|---|---|---|
| Example | (uM) | IC₅₀ (uM) |
| 1 | 0.002 | 0.012 |
| 2 | 0.003 | 0.012 |
| 3 | 0.002 | 0.015 |
| 4 | 0.014 | 0.119 |
| 5 | 0.070 | 0.322 |
| 6 | 0.004 | 0.024 |

TABLE 5

| | WT IC₅₀ | M184V |
|---|---|---|
| Example | (μM) | IC₅₀ (μM) |
| 1 | 0.001 | 0.010 |
| 2 | 0.003 | 0.015 |
| 3 | 0.003 | 0.014 |
| 4 | 0.020 | 0.111 |
| 5 | 0.067 | 0.333 |
| 6 | 0.004 | 0.021 |
| 7 | 0.014 | 0.055 |
| 8 | 0.008 | 0.039 |
| 9 | 0.005 | 0.026 |
| 10 | 0.003 | 0.019 |
| 11 | 0.003 | 0.013 |
| 12 | 0.002 | 0.009 |
| 13 | 0.002 | 0.012 |
| 14 | 0.002 | 0.011 |
| 15 | 0.004 | 0.026 |
| 16 | 2.280 | 10.740 |
| 17 | 0.003 | 0.013 |
| 18 | 0.002 | 0.014 |
| 19 | 0.030 | 0.150 |
| 20 | 0.025 | 0.091 |
| 21 | 0.025 | 0.113 |
| 22 | 0.036 | 0.176 |
| 23 | 0.001 | 0.007 |
| 24 | 0.008 | 0.037 |
| 25 | 0.021 | 0.090 |

### Antiviral Persistence Assay

The PSV assay was adapted to determine the antiviral persistence of each compound. This assay evaluates the ability of each compound to remain active in cells for two days i.e prevent PSV infection of cells in a dose dependent manner, 48 h after the removal of compound. Duplicate plates of U373 cells were treated with a serial dilution of small molecule inhibitors for 6 h at 37° C. Compounds were removed from cells by washing twice cells with 1×PBS. For baseline group (i.e immediately after washing or 0 h), cells were infected with prepared PSVs and cultured for three days. For experimental group (48 h), the culture medium is added to the washed cells and the plate incubated at 37° C. for 48 h. After two days of culture, the prepared PSVs were added to the cells and the mixture cultured for three days. The amount of luciferase signal produced after culture was used to reflect the level of pseudotyped virus infection in the baseline group (0 h) and experimental group (48 h) for each compound. An IC₅₀, or the concentration of inhibitor required to reduce PSV infection by 50% from the infection containing no inhibitor was calculated. The persistence index, which is the ratio of the IC₅₀ determined at 48 and 0 h is presented in Table 2 as well as the fold change of the persistence index relative to EFdA [(2R,3S,5R)-5-(6-amino-2-fluoro-9H-purin-9-yl)-2-ethynyl-2-(hydroxymethyl)tetrahydrofuran-3-ol].

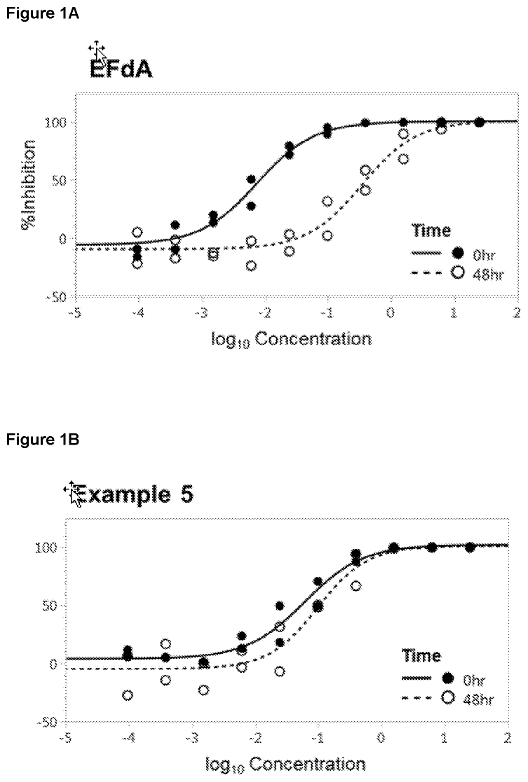

Statistical analysis and graphing of the data were performed in JMP 13.2.1 (SAS Institute, Cary, N.C.). A four-parameter logistic Hill Model was fit to % Inhibition and log₁₀ concentration values, separately for each compound, time point and run. A pilot experiment included 2 independent experimental runs and a later follow-up experiment included 4 runs. Quality control criteria based on R² and 95% confidence interval ranges of all four parameter estimates were used to exclude curves with poor fits. Using inverse prediction, log₁₀ concentrations were obtained that correspond to 50% Inhibition (log₁₀|C50*) and the log₁₀ persistence index was calculated for each compound and run using the following formula: log₁₀ persistence Index=log₁₀|C50*48 hrs log₁₀|C50*_{0 hrs}. Next, a linear mixed effects model was fit on log₁₀ persistence index values with a fixed effect for compound and a random effect for experimental run, followed by post hoc contrasts to compare the log₁₀ persistence index of the positive control EFdA to the log₁₀ persistence index of other test compounds. The estimated LSMeans and differences were then back-transformed via 10^{Estimate} to the original scale and reported as persistence index and fold change respectively. Raw p-values were reported. Antiviral persistence data for examples 1,4,5,6 and EFdA are shown in Table 6. IC₅₀ curve shifts from t=0 to t=48 h for EFdA and example 5 are illustrated in FIGS. 1A and 1B. The curves in FIGS. 1A and 1B were obtained from a single curve fit across replicate runs instead of separate fits for each run as described above.

TABLE 6

| | WT IC₅₀ | WT IC₅₀ | | Fold | |
|---|---|---|---|---|---|
| | (μM) at | (μM) at | Persistence | Change | |
| Example | t = 0 h | t = 48 h | Index | vs EFdA | p-Value |
| EFdA | 0.0074 | 0.3156 | 42.87 | 1.00 | — |
| 1 | 0.0025 | 0.0891 | 35.22 | 1.33 | 0.5826 |
| 4 | 0.0211 | 0.6906 | 32.78 | 1.31 | 0.6714 |
| 5 | 0.0517 | 0.1017 | 1.97 | 23.90 | 0.0066 |
| 6 | 0.0037 | 0.1797 | 56.90 | 0.75 | 0.6784 |

1. A compound of the formula (I): 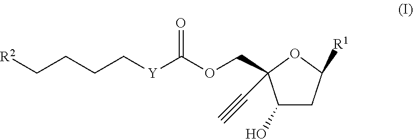
wherein:
R₁ is selected from the group consisting of: 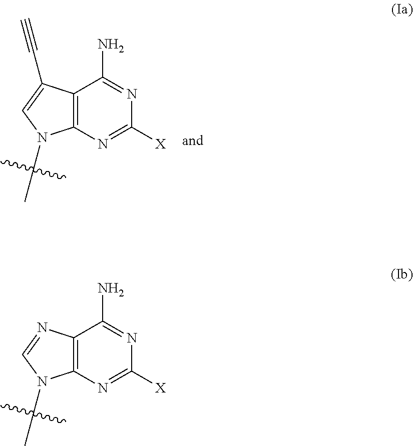
wherein:
X is selected from the group consisting of NH₂, F and Cl;
Y is selected from the group consisting of a bond, (C₁-C₁₀) alkyl and CR³R^{3′}, wherein R³ and R^{3′} are independently selected from the group consisting of H, (C₁-C₆) alkyl, (C₁-C₆) haloalkyl, (C₂-C₁₀) alkenyl, (C₁-C₁₀) alkynyl and (C₃-C₁₄) cycloalkyl; and each of R³ and R^{3′} may be independently optionally substituted by (C₁-C₆) alkyl, Cl, F, oxo, or (C₁-C₆) alkoxy
R² is selected from the group consisting of H, (C₁-C₁₀) alkyl, (C₂-C₁₀) alkenyl, (C₂-C₁₀) alkynyl, (C₁-C₁₀) alkoxy and (C₁-C₁₀) haloalkyl; wherein each of R² may be optionally substituted by (C₁-C₆) alkyl, Cl, F, oxo, or (C₁-C₆) alkoxy
or a pharmaceutically acceptable salt thereof.

2. The compound according to claim 1, wherein R₁ is:

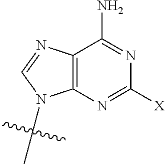

3. The compound according to claim 1, wherein X is F.

4. The compound according to claim 1, wherein Y is a bond and R² is H.

5. The compound according to claim 1, wherein Y is (C₁-C₁₀) alkyl and R² is (C₁-C₁₀) alkyl.

6. The compound according to claim 1, wherein Y is (C₁-C₁₀) alkyl and R² is (C₁-C₁₀) alkenyl.

7. The compound according to claim 1, wherein R² is (C₁-C₁₀) alkyl.

8. A compound of the formula: 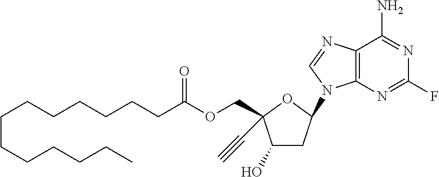 [SMILES: C#C[C@]1(COC(=O)CCCCCCCCCCCCC)O[C@@H](n2cnc3c(N)nc(F)nc32)C[C@@H]1O]
or a pharmaceutically acceptable salt thereof.

9. A compound selected from the group consisting of:

| Parent Structure | Chemical Name |
|---|---|
| 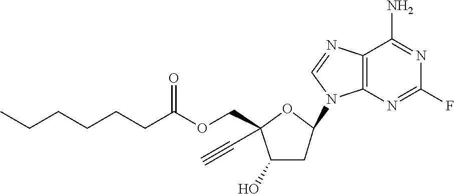 [SMILES: C#C[C@]1(COC(=O)CCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl heptanoate |
| 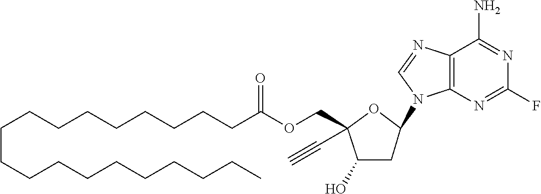 [SMILES: C#C[C@]1(COC(=O)CCCCCCCCCCCCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl icosanoate |
| 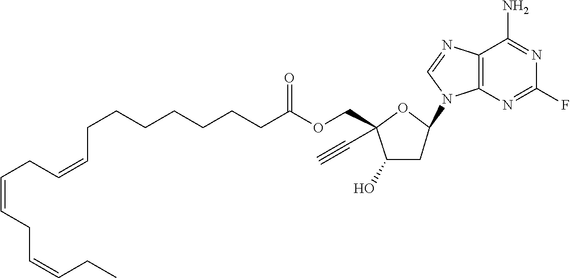 [SMILES: C#C[C@]1(COC(=O)CCCCCCC/C=C\C/C=C\C/C=C\CC)O[C@@H](n2cnc3c(N)nc(F)nc32)C[C@@H]1O] | (9Z,12Z,15Z)-((2R,3S,5R)-5-(6- amino-2-fluoro-9H-purin-9-yl)-2- ethynyl-3- hydroxytetrahydrofuran-2- yl)methyl octadeca-9,12,15- trienoate |
| 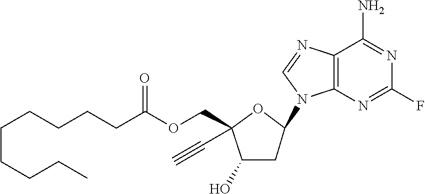 [SMILES: C#C[C@]1(COC(=O)CCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl decanoate |
| 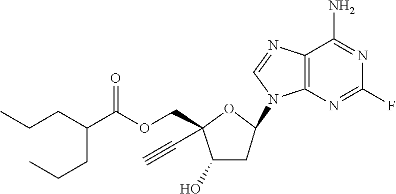 [SMILES: C#C[C@]1(COC(=O)C(CCC)CCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl 2-propylpentanoate |
| 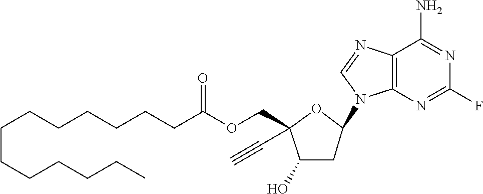 [SMILES: C#C[C@]1(COC(=O)CCCCCCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl tetradecanoate |
| 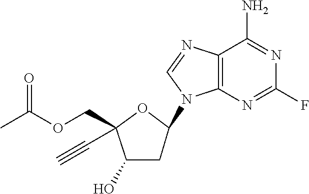 [SMILES: C#C[C@]1(COC(C)=O)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl acetate |
| 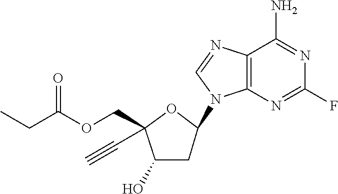 [SMILES: C#C[C@]1(COC(=O)CC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl propanoate |
| 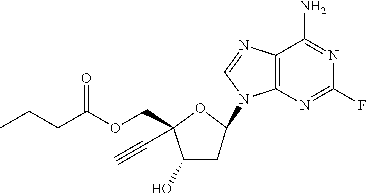 [SMILES: C#C[C@]1(COC(=O)CCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl butanoate |
| 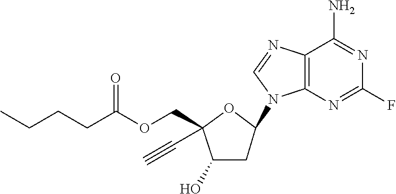 [SMILES: C#C[C@]1(COC(=O)CCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl pentanoate |
| 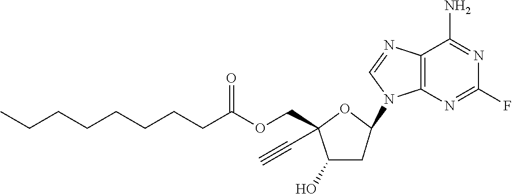 [SMILES: C#C[C@]1(COC(=O)CCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl nonanoate |
| 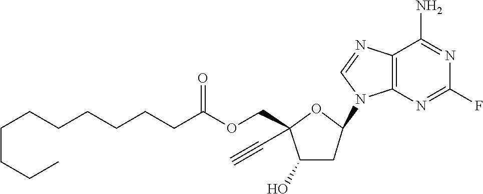 [SMILES: C#C[C@]1(COC(=O)CCCCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl undecanoate |
| 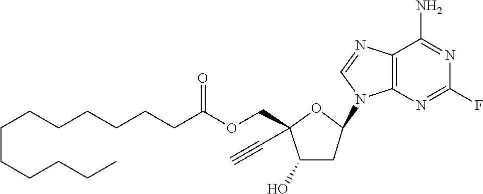 [SMILES: C#C[C@]1(COC(=O)CCCCCCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl tridecanoate |
| 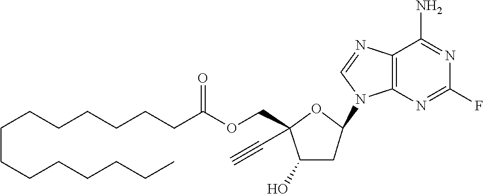 [SMILES: C#C[C@]1(COC(=O)CCCCCCCCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl pentadecanoate |
| 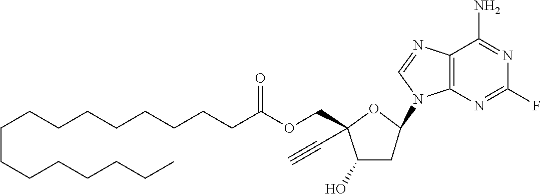 [SMILES: C#C[C@]1(COC(=O)CCCCCCCCCCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl heptadecanoate |
| 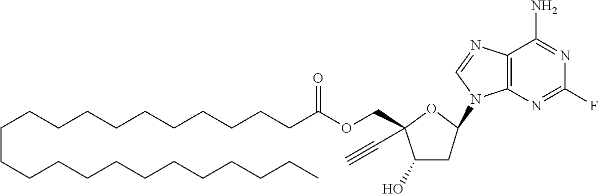 [SMILES: C#C[C@]1(COC(=O)CCCCCCCCCCCCCCCCCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl tetracosanoate |
| 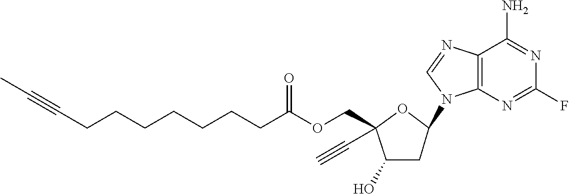 [SMILES: C#C[C@]1(COC(=O)CCCCCCCC#CC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl undec-9- ynoate |
| 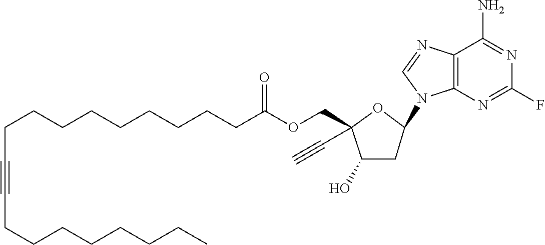 [SMILES: C#C[C@]1(COC(=O)CCCCCCCCCC#CCCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl octadec- 9-ynoate |
| 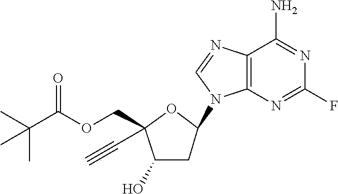 [SMILES: C#C[C@]1(COC(=O)C(C)(C)C)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl 2,2- dimethylpropanoate |
| 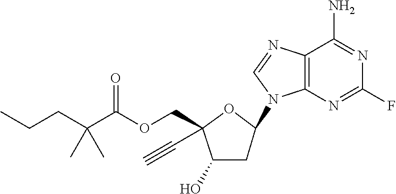 [SMILES: C#C[C@]1(COC(=O)C(C)(C)CCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl 2,2- dimethylpentanoate |
| 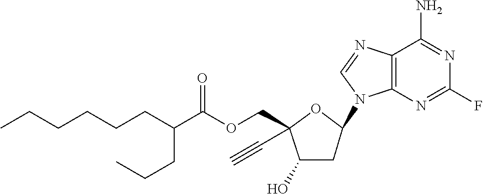 [SMILES: C#C[C@]1(COC(=O)C(CCC)CCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl 2- butyloctanoate |
| 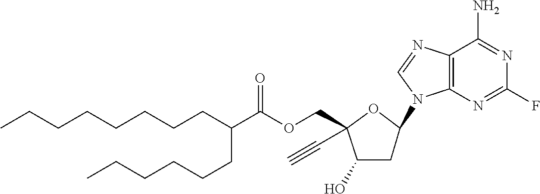 [SMILES: C#C[C@]1(COC(=O)C(CCCCCC)CCCCCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl 2- hexyldecanoate |
| 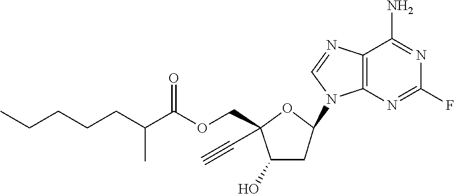 [SMILES: C#C[C@]1(COC(=O)C(C)CCCCC)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl 2- methylheptanoate |
| 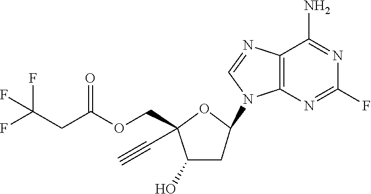 [SMILES: C#C[C@]1(COC(=O)CC(F)(F)F)O[C@@H](n2cnc3c(N)nc(F)nc32)C[C@@H]1O] | [(2R,3S,5R)-5-(6-amino-2-fluoro- 9H-purin-9-yl)-2-ethynyl-3- hydroxyoxolan-2-yl]methyl 3,3,3- trifluoropropanoate |
| | [(2R,3S,5R)-5-(6-amino-2-fluoro- |
| | 9H-purin-9-yl)-2-ethynyl-3- |
| | hydroxyoxolan-2-yl]methyl 3,3,3- |
| | trifluoro-2,2-dimethylpropanoate |

and pharmaceutically acceptable salts thereof.

10. A pharmaceutical composition comprising a compound according to claim 1, or a pharmaceutically acceptable salt thereof, and a pharmaceutically acceptable excipient.

11. The composition of claim 10, wherein the composition is present in parenteral form.

12. The composition of claim 10, wherein the composition is in a tablet form.

13. A method of treating an HIV infection in a subject comprising administering to the subject a compound of claim 1, or a pharmaceutically acceptable salt thereof.

14. (canceled)

15. A method of preventing an HIV infection in a subject at risk for developing an HIV infection, comprising administering to the subject a compound of claim 1, or a pharmaceutically acceptable salt thereof.

16-20. (canceled)
